SEQUENCE LISTING (7 sequences)

SEQ ID NO: 1 - DNA, 51 bp, Artificial Sequence
  Tacman Probe
ggggcctacacggatggccacagccrtcgcccgctgcgccccagatgagga

SEQ ID NO: 2 - DNA, 51 bp, Artificial Sequence
  Tacman Probe
agcactacaggcagcaccagcgaagrggccgtgacagccgttgccatctgc

SEQ ID NO: 3 - DNA, 51 bp, Artificial Sequence
  Tacman Probe
aaggatatagggaaaaccttgaaagygatgtctgtggtggccgtctttggc

SEQ ID NO: 4 - DNA, 51 bp, Artificial Sequence
  Taqman Probe
tacgaggagctggtgctagccttgckttccgaggaggacggcctggccgaa

SEQ ID NO: 5 - DNA, 51 bp, Artificial Sequence
  Tacman Probe
ccgtgacagccgttgccatctgctgmcggagccggcacctggcgcaggcct

SEQ ID NO: 6 - DNA, 51 bp, Artificial Sequence
  Tacman Probe
ggtactgacccccaacctggtggccrccctgccccccagcacccatggggc

SEQ ID NO: 7 - DNA, 51 bp, Artificial Sequence
  Tacman Probe
gagtgtggcctgtgcagaagggaccraggctggtgagaccaggagggcctg

(19) United States
(12) Patent Application Publication
(10) Pub. No.: US 2017/0183712 A1
(43) Pub. Date: Jun. 29, 2017

(54) METHODS OF ISOLATION OF CELL FREE COMPLEXES AND CIRCULATING CELL-FREE NUCLEIC ACID

(71) Applicant: ATHEROTECH, INC., Birmingham, AL (US)

(72) Inventor: Chen-Hsiung YEH, Birmingham, AL (US)

(21) Appl. No.: 15/313,006

(22) PCT Filed: May 21, 2015
(86) PCT No.: PCT/US15/32020

§ 371 Date: Nov. 21, 2016

Related U.S. Application Data
(60) Provisional application No. 62/000,941, filed on May 20, 2014.

(51) Int. Cl. C12Q 1/68

(52) U.S. Cl. CPC C12Q 1/6806

(57) ABSTRACT

The present disclosure provides methods for the isolation of a cell free complex comprising a circulating, cell free nucleic acid component. The nucleic acid component of the complex is, at least in part, associated with cellular components, such as polypeptides and lipids. The methods of the present disclosure provide for the isolation of circulating cell free nucleic acids that are longer and less fragmented as compared to prior art methods. Furthermore, the circulating, cell free nucleic acid represent biologically relevant nucleic acid targets as the methods described selectively remove non-functional nucleic acids from the isolated material. The nucleic acid component of the isolated complexes reflects a wide spectrum of genomic representation enabling successful detection of mutations, polymorphisms, methylation status and other genomic markers for use in diagnostic and therapeutic applications. Furthermore, the additional cellular components in the cell free complexes isolated provide information regarding the proteomic and lipidomic status of the subject

### BACKGROUND

The majority of nucleic acids (DNA and RNA) in the body are located within cells. However, extracellular nucleic acids can also be found circulating in the bloodstream. Such circulating, cell free nucleic acid may be the result of active, spontaneous release of newly synthesized nucleic acids from the cell or the result of release from necrotic and apoptotic cell death (Stroun M, et al., (2001) Clin Chim Acta 313: 139-142; van der Vaart M, et al., (2008) Ann N Y Acad Sci 1137: 18-26).

Circulating, cell free nucleic acid offers an unprecedented non-invasive approach to a wide range of diagnostics for clinical disorders not only in predictive and proactive medicine but also in personalized medicine. Further, circulating, cell free nucleic acid can offer unique opportunities for early surveillance of disease onset, e.g. in early cancer detection (Gormally E, et al., (2004) Int J Cancer 111: 746-749; Fleischhacker M, et al., (2007) Biochim Biophys Acta 1775: 181-232; Frattini M, et al., (2008) Cancer Lett 263: 170-181; Schwarzenbach H, et al., (2008) Ann N Y Acad Sci 1137: 190-196). The ability to isolate, quantify, and analyze circulating, cell free nucleic acid has led to the identification of disease-specific aberrations such as chromosomal abnormalities, gene mutations, methylation and copy number variations which are indicative of diseased cells (Diehl F, et al., (2005) Proc Natl Acad Sci USA 102: 16368-16373; Allegra C J, et al., (2009) J Clin Oncol 27: 2091-2096; Sunami, E.; et al., (2009) Methods Mol. Biol. 507, 349-356; Lu, Y. et al., (2013) PLoS One 2013, 8, e63056). It is believed that over the next decade circulating, cell free nucleic acid will become a non-invasive, standard-of-care for the determination of molecular markers in cancer management. Of particular interest is the growing belief that the analysis of circulating, cell free nucleic acid may provide a more global picture beyond the abnormalities and heterogeneity presented in the primary tumor tissue.

Circulating, cell free nucleic acid can be used as a disease biomarker in a number of ways. Increases and/or decreases in concentration of circulating, cell free nucleic acid may indicate the presence of a disease or the predisposition to a certain disease. Furthermore, the presence of tumor-specific mutations, gene expression signatures or other genomic signatures (for example, methylation patters) may also indicate the presence of a disease or the predisposition to a disease. In addition, the overall expression profile may also be used. Although a number of methods for extraction of circulating, cell free nucleic acid are known, each of the methods of the prior art suffer from certain deficiencies. Generally, the efficiency and quantification of circulating, cell free nucleic acid by the methods of the prior art is variable due to lack of normalization of the experimental conditions. Further, a significant portion of circulating, cell free nucleic acid obtained by the methods of the prior art is highly fragmented with the average size of fragments ranging 100-500 bp in the case of DNA (Mouliere F, Lu, Y. et al., (2013) PLoS One 2013, 8, e63056 (2011) PLoS ONE 6(9): e23418). Importantly, the methods of the prior art isolate free nucleic acids that are not associated with additional cellular components. Such an approach is less than optimal as the removal of co-associating cellular components represents a loss of valuable information. Such approaches also fail to target nucleic acids that are associated with polypeptide and other components that indicate the isolated nucleic acids are active physiologically and relevant to disease diagnosis and treatment. Importantly, nucleic acids associated with other cellular components are protected from degradation in the circulation, allowing the isolation of longer nucleic acids containing more information.

Accordingly, it is desirable to develop novel technologies that can consistently and selectively enrich, in high yield and purity, protected, higher-molecular-weight, biologically relevant circulating, cell free nucleic acid and associated cellular components. Such circulating cell free nucleic acid and associated cellular components may then be used in a variety of analytic, diagnostic and other approaches to indentify genomic, proteomic and lipidomic characteristics for disease diagnosis, prognosis, treatment and monitoring. The present disclosure provides such methods.

### BRIEF DESCRIPTION OF THE FIGURES

FIG. 1 shows a simplified flow chart illustrating the steps performed in one embodiment of the methods described.

FIG. 1B shows two exemplary chromatograms on the material isolated from plasma samples obtained from a subject using the methods described. Chromatograms were obtained by monitoring ultraviolet absorbance at 260 nm.

FIG. 2 shows chromatograms (obtained by monitoring ultraviolet absorbance at 260 nm) illustrating the effect of pre-treatment of the plasma samples with DNase, DNase and RNase and proteinase K before density gradient centrifugation by the methods described herein.

FIG. 3 shows agarose gels illustrating the effect of pre-treatment of the plasma samples with DNase, DNase and RNase, mung bean nuclease and proteinase K before density gradient centrifugation by the methods described herein.

FIG. 4 shows agarose gels of circulating cell free DNA isolated using the methods of the present disclosure and two commercially available extraction kits.

FIG. 5 shows representative results of array comparative genome hybridization of select individual chromosomes using circulating cell free DNA isolated using the methods of the present disclosure as well as comparing such detection using circulating cell free DNA isolated using the commercially available Kit Q of Example 4 and reference genomic DNA.

FIG. 6 shows the results of array comparative genome hybridization of each autosome and allosome using circulating cell free DNA isolated using the methods of the present disclosure as well as comparing such detection using circulating cell free DNA isolated using the commercially available Kit Q of Example 4 and reference genomic DNA.

FIG. 7 shows the identification of lipid components associated with the isolated circulating cell free nucleic acid.

### SUMMARY OF THE DISCLOSURE

In a first aspect, the present disclosure provides a method for the isolation of a cell free complex comprising circulating, cell-free nucleic acid and associated cellular components, the method comprising subjecting a sample containing the cell free complex to centrifugation and isolating the cell free complex. Such method may further comprise isolating at least one cell-free nucleic acid from the complex, the nucleic acid optionally associated with a cellular component, such as, but not limited to, a polypeptide and/or a lipid. Such method may further comprise isolating at least cellular component from the complex. In one instance, the cellular component is a lipid or a protein. Such nucleic acid may be DNA and/or RNA.

In a second aspect, the present disclosure provides a method for the isolation of a cell free complex comprising circulating, cell-free nucleic acid and associated cellular components, the method comprising subjecting a sample containing the cell free complex to density gradient centrifugation and isolating the cell free complex from the gradient. Such method may further comprise isolating at least one cell-free nucleic acid from the complex, the nucleic acid optionally associated with a cellular component, such as, but not limited to, a polypeptide and/or a lipid. Such method may further comprise isolating at least cellular component from the complex. In one instance, the cellular component is a lipid or a protein. Such nucleic acid may be DNA and/or RNA.

In a third aspect, the present disclosure provides a method for the isolation of a cell free complex comprising circulating, cell-free nucleic acid and associated cellular components, the method comprising subjecting a sample containing the cell free complex to density gradient centrifugation with a buoyant density of 1.3 to 1.45 g/cm³ and isolating the cell free complex from the gradient. Such method may further comprise isolating at least one cell-free nucleic acid from the complex, the nucleic acid optionally associated with a cellular component, such as, but not limited to, a polypeptide and/or a lipid. Such method may further comprise isolating at least cellular component from the complex. In one instance, the cellular component is a lipid or a protein. Such nucleic acid may be DNA and/or RNA.

In a fourth aspect, the present disclosure provides a method for the isolation of a cell free complex comprising circulating, cell-free nucleic acid and associated cellular components, the method comprising subjecting a sample containing the cell free complex to cesium-chloride (CsCl) density gradient centrifugation with a buoyant density of 1.3 to 1.45 g/cm³ and isolating the cell free complex from the gradient. Such method may further comprise isolating at least one cell-free nucleic acid from the complex, the nucleic acid optionally associated with a cellular component, such as, but not limited to, a polypeptide and/or a lipid. Such method may further comprise isolating at least cellular component from the complex. In one instance, the cellular component is a lipid or a protein. Such nucleic acid may be DNA and/or RNA.

In a fifth aspect, the present disclosure provides a method for the detection of a cell free complex comprising circulating, cell-free nucleic acid and associated cellular components, the method comprising isolating the cell free complex as set forth in any one of the first through fourth aspects, optionally further processing the cell free complex and associated cellular components and detecting the cell-free nucleic acid, the associated cellular component or a combination of the foregoing. Such cellular components include, but are not limited to, polypeptides and lipids. Such nucleic acid may be DNA and/or RNA.

In a sixth aspect, the present disclosure provides a method for analysis of a cell free complex comprising circulating, cell-free nucleic acid and associated cellular components, the method comprising isolating the cell free complex as set forth in any one of the first through fourth aspects, optionally further processing the cell free complex and analyzing the cell-free nucleic acid, associated cellular components or a combination of the foregoing to determine a characteristic of the nucleic acid, associated cellular components or a combination of the foregoing. Such cellular components include, but are not limited to, polypeptides and lipids. Such nucleic acid may be DNA and/or RNA.

In a seventh aspect, the present disclosure provides a method for analysis of a cell free complex comprising circulating, cell-free nucleic acid and associated cellular components, the method comprising isolating the cell free complex as set forth in any one of the first through fourth aspects, optionally further processing the cell free complex and analyzing the cell-fee nucleic acid to determine a nucleic acid characteristic. The method may further comprise analyzing a cellular component. Such additional cellular components include, but are not limited to, polypeptides and lipids. Such nucleic acid may be DNA and/or RNA.

In an eighth aspect, the present disclosure provides a method for analysis of a cell free complex comprising circulating, cell-free nucleic acid and associated cellular components, the method comprising isolating the cell free complex as set forth in any one of the first through fourth aspects, optionally further processing the cell free complex and analyzing the associated polypeptide component to determine a polypeptide characteristic. The method may further comprise analyzing a nucleic acid component and/or an additional cellular component. Such additional cellular components include, but are not limited to, lipids. Such nucleic acid may be DNA and/or RNA.

In a ninth aspect, the present disclosure provides a method for analysis of a cell free complex comprising circulating, cell-free nucleic acid and associated cellular components, the method comprising isolating cell free complex as set forth in any one of the first through fourth aspects, optionally further processing the cell free complex and analyzing the associated lipid component to determine a lipid characteristic. The method may further comprise analyzing a nucleic acid component and/or an additional cellular component. Such cellular additional components include, but are not limited to, polypeptides. Such nucleic acid may be DNA and/or RNA.

In tenth aspect, the present disclosure provides a method for analysis of a cell free complex comprising circulating, cell-free nucleic acid and associated cellular components, the method comprising isolating the cell free complex as set forth in any one of the first through fourth aspects, optionally further processing the cell free complex and analyzing the cell-free nucleic acid to determine the presence of a mutation, the presence of a polymorphism, the methylation state, the concentration of a nucleic acid, the level of expression of a nucleic acid or a combination of the foregoing. The method may further comprise analyzing a cellular component. Such cellular components include, but are not limited to, polypeptides and lipids. Such nucleic acid may be DNA and/or RNA.

In an eleventh aspect, the present disclosure provides a method for determining a biologically relevant profile of a subject, the method comprising isolating a cell free complex comprising circulating, cell-free nucleic acid and associated cellular components as set forth in any one of the first through fourth aspects, optionally further processing the cell free complex and identifying a plurality of nucleic acids or associated cellular components to produce the profile. Such method may further comprise comparing the subject profile to a corresponding profile indicative of a disease state and determining the subject is suffering from or at risk for a particular disease or condition if the subject profile contains one or more characteristics of the corresponding profile indicative of a disease state. The profile may be a nucleic acid profile, a polypeptide profile, a lipid profile or a combination of the foregoing. Such nucleic acid may be DNA and/or RNA.

In a twelfth aspect, the present disclosure provides a method for determining a biologically relevant nucleic acid profile of a subject, the method comprising isolating a cell free complex comprising circulating, cell-free nucleic acid and associated cellular components as set forth in any one of the first through fourth aspects, optionally further processing the cell free complex and identifying a plurality of cell-free nucleic acids to produce the nucleic acid profile. Such method may further comprise comparing the subject profile to a corresponding nucleic acid profile indicative of a disease state and determining the subject is suffering from or at risk for a particular disease or condition if the subject profile contains one or more characteristics of the corresponding nucleic acid profile indicative of a disease state.

In a thirteenth aspect, the present disclosure provides a method for determining a biologically relevant polypeptide profile of a subject, the method comprising isolating a cell free complex comprising circulating, cell-free nucleic acid and associated cellular components as set forth in any one of the first through fourth aspects, optionally further processing the cell free complex and identifying a plurality of polypeptides in the associated cellular components to produce a polypeptide profile. Such method may further comprise comparing the subject profile to a corresponding polypeptide profile indicative of a disease state and determining the subject is suffering from or at risk for a particular disease or condition if the subject profile contains one or more characteristics of the corresponding polypeptide profile indicative of a disease state.

In a fourteenth aspect, the present disclosure provides a method for determining a biologically relevant lipid profile of a subject, the method comprising isolating a cell free complex comprising circulating, cell-free nucleic acid and associated cellular components as set forth in any one of the first through fourth aspects, optionally further processing the cell free complex and identifying a plurality of lipids in the isolated associated cellular components to produce a lipid profile. Such method may further comprise comparing the subject profile to a corresponding lipid profile indicative of a disease state and determining the subject is suffering from or at risk for a particular disease or condition if the subject profile contains one or more characteristics of the corresponding lipid profile indicative of a disease state.

In a fifteenth aspect, the present disclosure provides a method for determining if a subject is suffering from or at risk for a disease, the method comprising isolating a cell free complex comprising circulating, cell-free nucleic acid and associated cellular components as set forth in any one of the first through fourth aspects, optionally further processing the cell free complex and determining the presence of a characteristic associated with the disease in the components of the complex. Such characteristic may be the presence of nucleic acid characteristic, a proteomic characteristic or a lipid characteristic.

In a sixteenth aspect, the present disclosure provides a method for determining if a subject is suffering from or at risk for a disease, the method comprising isolating a cell free complex comprising circulating, cell-free nucleic acid and associated cellular components as set forth in any one of the first through fourth aspects, optionally further processing the cell free complex and determining the presence of a nucleic acid characteristic in the cell-free nucleic acid, wherein the subject is judged to be suffering from or at risk for a disease if the presence of any of the characteristic is associated with the disease. Such nucleic acid characteristic includes, but is not limited to, a mutation, a polymorphism, methylation state, concentration of a nucleic acid, level of expression of a nucleic acid, the profile of the nucleic acids or a combination of the foregoing.

In a seventeenth aspect, the present disclosure provides a method for determining if a subject is suffering from or at risk for a disease, the method comprising isolating a cell free complex comprising circulating, cell-free nucleic acid and associated cellular components as set forth in any one of the first through fourth aspects, optionally further processing the cell free complex and determining the presence of a polypeptide and/or lipid characteristic in the cellular component, wherein the subject is judged to be suffering from or at risk for a disease if the presence of any of the characteristic is associated with the disease. Such polypeptide characteristic includes, but is not limited to, a mutation, the presence of post-translational modifications, the presence of insertions or deletions, the concentration, level of expression of a nucleic acid, the profile of the polypeptides or a combination of the foregoing. Such lipid characteristic includes, but is not limited to, the presence of altered forms, the presence of modifications, the concentration, the expression level, the profile of lipids or a combination of the foregoing.

In an eighteenth aspect, the present disclosure provides a method for determining if a subject is suffering from or at risk for a disease, the method comprising generating a nucleic acid, polypeptide and/or lipid profile from a subject as set forth in the twelfth to fourteenth aspects and comparing the generated profile to a corresponding disease fingerprint and determining the subject is suffering from or at risk for a particular disease or condition if the subject profile contains one or more characteristics of the disease fingerprint.

In an nineteenth aspect, the present disclosure provides a method for the isolation of circulating, cell-free nucleic acid, the method comprising subjecting a sample containing the circulating, cell-free nucleic acid to centrifugation and isolating the circulating, cell-free nucleic acid. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In a twentieth aspect, the present disclosure provides a method for the isolation of circulating, cell-free nucleic acid, the method comprising subjecting a sample containing the to density gradient centrifugation and isolating the circulating, cell-free nucleic acid from the gradient. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In a twenty-first aspect, the present disclosure provides a method for the isolation of circulating, cell-free nucleic acid, the method comprising subjecting a sample containing the circulating, cell free nucleic acid to density gradient centrifugation with a buoyant density of 1.3 to 1.45 g/cm³ and isolating the circulating, cell free nucleic acid from the gradient. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In a twenty-second aspect, the present disclosure provides a method for the isolation of the circulating, cell-free nucleic acid, the method comprising subjecting a sample containing the cell free complex to cesium-chloride (CsCl) density gradient centrifugation with a buoyant density of 1.3 to 1.45 g/cm³ and isolating the circulating, cell-free nucleic acid from the gradient. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In a twenty-third aspect, the present disclosure provides a method for the detection of circulating, cell-free nucleic acid, the method comprising isolating the circulating, cell-free nucleic acid as set forth in any one of the nineteenth to twenty-second aspects, optionally further processing the circulating, cell-free nucleic acid and detecting the circulating, cell-free nucleic acid. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and detecting the cellular component. In one instance, the cellular component is a lipid or a protein. Such nucleic acid may be DNA and/or RNA.

In a twenty-fourth aspect, the present disclosure provides a method for analysis of circulating, cell-free nucleic acid, the method comprising isolating the circulating, cell-free nucleic acid as set forth in any one of the nineteenth to twenty-second aspects, optionally further processing the circulating, cell-free nucleic acid and analyzing the circulating, cell-free nucleic acid to determine a characteristic of the nucleic acid. Such nucleic acid characteristic includes, but is not limited to, a mutation, a polymorphism, methylation state, concentration of a nucleic acid, level of expression of a nucleic acid, the profile of the nucleic acids or a combination of the foregoing. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In an twenty-fifth aspect, the present disclosure provides a method for analysis of circulating, cell-free nucleic acid, the method isolating the circulating, cell-free nucleic acid as set forth in any one of the nineteenth to twenty-second aspects, optionally further processing the circulating, cell-free nucleic acid and analyzing the circulating, cell-free nucleic acid to determine the presence of a mutation, the presence of a polymorphism, the methylation state, the concentration of a nucleic acid, the level of expression of a nucleic acid or a combination of the foregoing. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In a twenty-sixth aspect, the present disclosure provides a method for determining a biologically relevant nucleic acid profile of a subject, the method comprising isolating circulating, cell-free nucleic acid as set forth in any one of the nineteenth to twenty-second aspects, optionally further processing the circulating, cell-free nucleic acid and identifying a plurality of nucleic acids to produce the nucleic acid profile. Such method may further comprise comparing the subject profile to a corresponding nucleic acid profile indicative of a disease state and determining the subject is suffering from or at risk for a particular disease or condition if the subject profile contains one or more characteristics of the corresponding nucleic acid profile indicative of a disease state. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In a twenty-seventh aspect, the present disclosure provides a method for determining if a subject is suffering from or at risk for a disease, the method comprising isolating circulating, cell-free nucleic acid as set forth in any one of the nineteenth to twenty-second aspects, optionally further processing the cell-free nucleic acid and determining the presence of a nucleic acid characteristic, wherein the subject is judged to be suffering from or at risk for a disease if the presence of any of the characteristic is associated with the disease. Such nucleic acid characteristic includes, but is not limited to, a mutation, a polymorphism, methylation state, concentration of a nucleic acid, level of expression of a nucleic acid, the profile of the nucleic acids or a combination of the foregoing. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In a twenty-eight aspect, the present disclosure provides a method for determining if a subject is suffering from or at risk for a disease, the method comprising generating a nucleic acid profile from a subject as set forth in the twenty-sixth aspect and comparing the generated profile to a corresponding disease fingerprint and determining the subject is suffering from or at risk for a particular disease if the subject profile contains one or more characteristics of the disease fingerprint. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA

### DETAILED DESCRIPTION

### Definitions

As used herein the term “cell free complex” refers to a complex comprising a cell free nucleic acid component and at least one cellular component. Such cellular components include but are not limited to, polypeptides, proteins and lipids. The cell free complex may contain a nucleic acid component, a polypeptide component and a lipid component or a nucleic acid component and one of a polypeptide component and a lipid component. For clarity, while the term “cell free complex” requires the presence of a nucleic acid component and at least one cellular component, the term also includes nucleic acids that are not associated with a cellular component provided that at least a portion of the nucleic acids in the cell free complex are associated with at least one additional cellular component.

As used herein, the term “genomic characteristic” or “nucleic acid characteristic” refers to a characteristic of the nucleic acid contained in the cell free complex. Any characteristic that is used to provide information regarding the nucleic acid may be used. Such characteristics include, but are not limited to, the presence of mutations, the presence of polymorphisms, the methylation pattern, the concentration, the expression level and the profile of nucleic acids contained in the cell free complex or associated with the cell-free nucleic acid. The foregoing characteristics may be determined by comparing to a wild-type counterpart of the nucleic acid.

As used herein, the term “proteomic characteristic” or “polypeptide characteristic” refers to a characteristic of the polypeptides contained in the cell free complex. Any characteristic that is used to provide information regarding the polypeptide may be used. Such characteristics include, but are not limited to, the presence of mutations, the presence of post-translational modifications (for example, phosphorylation), the presence of insertions or deletions, the concentration, the expression level and the profile of polypeptides contained in the cell free complex or associated with the cell-free nucleic acid. The foregoing characteristics may be determined by comparing to a wild-type counterpart of the polypeptide.

As used herein, the term “lipidomic characteristic” or “lipid characteristic” refers to a characteristic of the lipids contained in the cell free complex. Any characteristic that is used to provide information regarding the lipid may be used. Such characteristics include, but are not limited to, the presence of altered forms (for example, carbon chain length), the presence of modifications (for example, changes in numbers of double and/or triple bonds), the concentration, the expression level and the profile of lipids contained in the cell free complex or associated with the cell-free nucleic acid. The foregoing characteristics may be determined by comparing to a wild-type counterpart of the lipid.

As used herein, the term “isolated” means the partial purification or increase in concentration of a component of a sample, such as the cell free complex or a component thereof. Such isolation does not require absolute purification. For example, when isolated is used in reference to a nucleic acid, the nucleic acid may comprise 50% or more, 60% or more, 70% or more, 80% or more, 90% or more, 95% or more of 99% or more of the material (by weight) in the mixture isolated.

As used herein, the term “subject” may be any mammal and in one embodiment is a human. The human subject may be any age. The human subject may also be an unborn child.

### Introduction

As discussed above, the present methods for isolating circulating, cell free nucleic acid suffer from several disadvantages. The present disclosure overcomes these disadvantages by providing novel centrifugation methods for the isolation of circulating, cell free nucleic acid and/or a cell free complex comprising a circulating, cell free nucleic acid component along with associated cellular components. When a cell free complex is isolated, the nucleic acid component of the complex, in one embodiment, is associated with cellular components, such as, but not limited to, polypeptides and lipids. In certain embodiment, the circulating cell free nucleic acid is associated with both polypeptides and lipids. Such association may be direct or indirect. However, the nucleic acid is not required to be physically associated with the cellular components. For example, the nucleic acid component may simply be located in the same density gradient as the cellular components. Furthermore, in certain embodiment, the circulating cell free nucleic acid may not be associated with a cellular component. The methods of the present disclosure provide for the isolation of circulating cell free nucleic acids that are longer and less fragmented as compared to prior art methods. In one embodiment, such a result is due, at least in part, to protection from degradation of the nucleic acid component by the associated cellular components. Furthermore, the circulating, cell free nucleic acid represent biologically relevant nucleic acid targets as the methods described selectively remove non-functional nucleic acids from the isolated material. The nucleic acid component of the isolated complexes reflects a wide spectrum of genomic representation enabling successful detection of mutations, polymorphisms, methylation status and other genomic markers for use in diagnostic and therapeutic applications. Furthermore, the additional cellular components in the cell free complexes isolated provide information regarding the proteomic and lipidomic status of the subject. Such markers are also useful in diagnostic and therapeutic applications. The resulting isolated cell free complex therefore provides a rich source of information regarding the genomic, proteomic and lipidomic status of a subject.

In principle, such centrifugation approaches requires no prior information about the cell free complexes, including the identity or composition of the nucleic acid, protein and lipid components, and is very sensitive since little starting material is needed. Most importantly, taking into account the fact that the vast majority of human genome doesn't code for proteins (over 98%), the selective enrichment provided by the methods of the present disclosure are superior to the methods of the prior art which rely on blind extraction methods that can lose up to 80% of circulating, cell free nucleic acids during extraction.

While not wishing to be bound by any particular theory, certain regions of nucleic acid (for example, enhancers, transcribed exons, or active promoters) are associated with cellular components during normal function. As a result, such nucleic acids have a density that is intermediate between free nucleic acid and protein. Therefore, density gradient centrifugation methods may be used to preferentially isolated cell free complexes that contain a nucleic acid component that is associated with additional cellular components.

### Methods of Isolating Cell Free Complexes and Components Therein

The methods of the present disclosure provide for the isolation of cell free complexes. Such cell free complexes comprise a cell free nucleic acid and at least one cellular component. Such cellular components include but are not limited to, polypeptides, proteins and lipids. The circulating, cell-free nucleic acids and cell free complexes isolated provide a biologically relevant, information rich source of information regarding the subject.

In one embodiment, the present disclosure provides a method for the isolation of a cell free complex comprising circulating, cell-free nucleic acid and associated cellular components, the method comprising subjecting a sample containing the cell free complex to centrifugation and isolating the cell free complex. Such method may further comprise isolating at least one cell-free nucleic acid from the complex, the nucleic acid optionally associated with a cellular component, such as, but not limited to, a polypeptide and/or a lipid.

In one embodiment, the present disclosure provides a method for the isolation of a cell free complex circulating, cell-free nucleic acid and associated cellular components, the method comprising subjecting a sample containing the cell free complex to density gradient centrifugation and isolating the cell free complex from the gradient. Such method may further comprise isolating at least one cell-free nucleic acid from the complex, the nucleic acid optionally associated with a cellular component, such as, but not limited to, a polypeptide and/or a lipid. Such method may further comprise isolating at least cellular component from the complex.

In one embodiment, the present disclosure provides a method for the isolation of a cell free complex comprising circulating, cell-free nucleic acid and associated cellular components, the method comprising subjecting a sample containing the cell free complex to density gradient centrifugation with a buoyant density of 1.3 to 1.45 g/cm³ and isolating the cell free complex from the gradient. Such method may further comprise isolating at least one cell-free nucleic acid from the complex, the nucleic acid optionally associated with a cellular component, such as, but not limited to, a polypeptide and/or a lipid. Such method may further comprise isolating at least cellular component from the complex.

In one embodiment, the present disclosure provides a method for the isolation of a cell free complex comprising circulating, cell-free nucleic acid and associated cellular components, the method comprising subjecting a sample containing the cell free complex to cesium-chloride (CsCl) density gradient centrifugation with a buoyant density of 1.3 to 1.45 g/cm³ and isolating the cell free complex from the gradient. Such method may further comprise isolating at least one cell-free nucleic acid from the complex, the nucleic acid optionally associated with a cellular component, such as, but not limited to, a polypeptide and/or a lipid. Such method may further comprise isolating at least cellular component from the complex.

In a particular embodiment of the foregoing, the density gradient is formed from CsCl. In a further particular embodiment, the CsCl density gradient has a density of between 1.30 and 1.45 g/cm³, 1.35 to 1.40 g/cm³, 1.35 to 1.45 g/cm³, 1.38 to 1.43 g/cm³ or 1.39 to 1.42 g/cm³. In a further particular embodiment, the nucleic acid is DNA, RNA or a combination of both DNA and RNA. Any form of known form of nucleic acid is included in the definition of nucleic acid.

In a particular embodiment, the length of the nucleic acid fragment isolated in the cell free complexes is over 500, over 750, over 1000, over 2000 or over 50000 base pairs. In another embodiment, the lent hog the nucleic acid fragments in the cell free complex is between 500 and 5000 base pairs, 1000 to 10,000 base pairs, 1000 to 5000 base pairs, or 10,000 to 20,000 base pairs.

In a particular embodiment of the foregoing, the nucleic acid is DNA, RNA or a combination of the foregoing. In a further particular embodiment, the nucleic acid is DNA. In a further particular embodiment, the nucleic acid is RNA.

In a particular embodiment of the foregoing, the associated cellular component is a lipid, a protein, a polypeptide or a combination of the foregoing. In a further particular embodiment, the cellular component is a lipid. In a further particular embodiment, the cellular component is a protein. In a further particular embodiment, the cellular component is a polypeptide.

In a particular embodiment of the foregoing, the cell free complex may be subject to additional processing steps, for example, to further isolate the cell-free nucleic acid and/or the cellular components from the cell free complex. Any means known in the art to accomplish such ends is included in the scope of the present disclosure.

### Methods of Isolating Circulating, Cell-Free Nucleic Acid

The methods of the present disclosure provide for the isolation of circulating, cell-free nucleic acids. Such nucleic acid may be DNA and/or RNA. The circulating, cell-free nucleic acids provide a biologically relevant, information rich source of information regarding the subject.

In one embodiment, the present disclosure provides a method for the isolation of a circulating, cell-free nucleic acid comprising subjecting a sample containing the circulating, cell-free nucleic acid to centrifugation and isolating the circulating, cell-free nucleic acid.

In another embodiment, the present disclosure provides a method for the isolation of a circulating, cell-free nucleic acid comprising subjecting a sample containing the circulating, cell-free nucleic acid to density gradient centrifugation and isolating the circulating, cell-free nucleic acid from the gradient.

In another embodiment, the present disclosure provides a method for the isolation of a circulating, cell-free nucleic acid comprising subjecting a sample containing the circulating, cell free nucleic acid to density gradient centrifugation with a buoyant density of 1.3 to 1.45 g/cm³ and isolating the circulating, cell free nucleic acid from the gradient.

In another embodiment, the present disclosure provides a method for the isolation of a circulating, cell-free nucleic acid comprising subjecting a sample containing the cell free complex to cesium-chloride (CsCl) density gradient centrifugation with a buoyant density of 1.3 to 1.45 g/cm³ and isolating the circulating, cell-free nucleic acid from the gradient.

In a particular embodiment of the foregoing, the circulating, cell-free nucleic acid may be present in a cell free complex and such method may further comprise isolating at least one cell-free nucleic acid from the complex. In a further particular embodiment of the foregoing, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid. In one instance, the cellular component is a lipid or a protein.

In a particular embodiment, the length of the nucleic acid fragment isolated in the cell free complexes is over 500, over 750, over 1000, over 2000 or over 50000 base pairs. In another embodiment, the lent hog the nucleic acid fragments in the cell free complex is between 500 and 5000 base pairs, 1000 to 10,000 base pairs, 1000 to 5000 base pairs, or 10,000 to 20,000 base pairs.

In a particular embodiment of the foregoing, the nucleic acid is DNA, RNA or a combination of the foregoing. In a further particular embodiment, the nucleic acid is DNA. In a further particular embodiment, the nucleic acid is RNA.

In a particular embodiment of the foregoing, the associated cellular component is a lipid, a protein, a polypeptide or a combination of the foregoing. In a further particular embodiment, the cellular component is a lipid. In a further particular embodiment, the cellular component is a protein. In a further particular embodiment, the cellular component is a polypeptide.

In a particular embodiment of the foregoing, the circulating cell free nucleic acid may be subject to additional processing steps, for example, to further isolate the cell-free nucleic acid from a cell free complex or associated cellular components. Any means known in the art to accomplish such ends is included in the scope of the present disclosure.

### Methods of Detection and Analysis of Cell Free Complexes

The present disclosure also provides methods for the detection and analysis of the circulating, cell-free nucleic acid and the components of the cell free complex isolated as described herein.

In one embodiment, the present disclosure provides a method for the detection of a cell free complex comprising circulating, cell-free nucleic acid and associated cellular components, the method comprising isolating the cell free complex as set forth in any one of the first through fourth aspects, optionally further processing the cell free complex and associated cellular components and detecting the cell-free nucleic acid, the associated cellular component or a combination of the foregoing.

In one embodiment, the present disclosure provides a method for analysis of a cell free complex comprising circulating, cell-free nucleic acid and associated cellular components, the method comprising isolating the cell free complex as set forth in any of the methods described herein, optionally further processing the cell free complex and analyzing the cell-free nucleic acid, associated cellular components or a combination of the foregoing to determine a characteristic of the nucleic acid, associated cellular components or a combination of the foregoing.

In one embodiment, the present disclosure provides a method for analysis of a cell free complex comprising circulating, cell-free nucleic acid and associated cellular components, the method comprising isolating the cell free complex as set forth in any of the methods described herein, optionally further processing the cell free complex and analyzing the cell-fee nucleic acid to determine a nucleic acid characteristic. The method may further comprise analyzing a cellular component.

In one embodiment, the present disclosure provides a method for analysis of a cell free complex comprising circulating, cell-free nucleic acid and associated cellular components, the method comprising isolating the cell free complex as set forth in any of the methods described herein, optionally further processing the cell free complex and analyzing the associated polypeptide component to determine a polypeptide characteristic. The method may further comprise analyzing a nucleic acid component and/or an additional cellular component.

In one embodiment, the present disclosure provides a method for analysis of a cell free complex comprising circulating, cell-free nucleic acid and associated cellular components, the method comprising isolating cell free complex as set forth in any of the methods described herein, optionally further processing the cell free complex and analyzing the associated lipid component to determine a lipid characteristic. The method may further comprise analyzing a nucleic acid component and/or an additional cellular component.

In one embodiment, the present disclosure provides a method for analysis of a cell free complex comprising circulating, cell-free nucleic acid and associated cellular components, the method comprising isolating the cell free complex as set forth in any of the methods described herein, optionally further processing the cell free complex and analyzing the cell-free nucleic acid to determine the presence of a mutation, the presence of a polymorphism, the methylation state, the concentration of a nucleic acid, the level of expression of a nucleic acid, the nucleic acid profile or a combination of the foregoing. The method may further comprise analyzing a cellular component.

In one embodiment, the present disclosure provides a method for analysis of a cell free complex comprising circulating, cell-free nucleic acid and associated cellular components, the method comprising isolating the cell free complex as set forth in any of the methods described herein, optionally further processing the cell free complex and analyzing the polypeptide component to determine the presence of a mutation, the presence of a post-translational modification, the presence of insertions or deletions, the concentration, level of expression of a nucleic acid, the profile of the polypeptides or a combination of the foregoing. The method may further comprise analyzing an additional cellular component or a nucleic acid.

In one embodiment, the present disclosure provides a method for analysis of a cell free complex comprising circulating, cell-free nucleic acid and associated cellular components, the method comprising isolating the cell free complex as set forth in any of the methods described herein, optionally further processing the cell free complex and analyzing the lipid component to determine the presence of altered forms, the presence of modifications, the concentration, the expression level, the profile of lipids or a combination of the foregoing.

In a particular embodiment of the foregoing, the nucleic acid is DNA, RNA or a combination of the foregoing. In a further particular embodiment, the nucleic acid is DNA. In a further particular embodiment, the nucleic acid is RNA.

In a particular embodiment, the length of the nucleic acid fragment isolated in the cell free complexes is over 500, over 750, over 1000, over 2000 or over 50000 base pairs. In another embodiment, the lent hog the nucleic acid fragments in the cell free complex is between 500 and 5000 base pairs, 1000 to 10,000 base pairs, 1000 to 5000 base pairs, or 10,000 to 20,000 base pairs.

In a particular embodiment of the foregoing, the associated cellular component is a lipid, a protein, a polypeptide or a combination of the foregoing. In a further particular embodiment, the cellular component is a lipid. In a further particular embodiment, the cellular component is a protein. In a further particular embodiment, the cellular component is a polypeptide.

In a particular embodiment of the foregoing, the circulating cell free nucleic acid may be subject to additional processing steps, for example, to further isolate the cell-free nucleic acid from a cell free complex or associated cellular components. Any means known in the art to accomplish such ends is included in the scope of the present disclosure.

### Methods of Detection and Analysis of Circulating Cell-Free Nucleic Acid

The present disclosure also provides methods for the detection and analysis of circulating, cell-free nucleic acid.

In one embodiment, the present disclosure provides a method for analysis of circulating, cell-free nucleic acid, the method comprising isolating the circulating, cell-free nucleic acid as set forth in any of the methods described herein, optionally further processing the circulating, cell-free nucleic acid and analyzing the circulating, cell-free nucleic acid to determine a characteristic of the nucleic acid. Such nucleic acid characteristic includes, but is not limited to, a mutation, a polymorphism, methylation state, concentration of a nucleic acid, level of expression of a nucleic acid, the profile of the nucleic acids or a combination of the foregoing.

In one embodiment, the present disclosure provides a method for analysis of circulating, cell-free nucleic acid, the method comprising isolating the circulating, cell-free nucleic acid as set forth in any of the methods described herein, optionally further processing the circulating, cell-free nucleic acid and analyzing the circulating, cell-free nucleic acid to determine the presence of a mutation, the presence of a polymorphism, the methylation state, the concentration of a nucleic acid, the level of expression of a nucleic acid or a combination of the foregoing

In a particular embodiment of the foregoing, the circulating, cell-free nucleic acid may be present in a cell free complex and such method may further comprise isolating at least one cell-free nucleic acid from the complex. In a further particular embodiment of the foregoing, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the at least one cellular component. In one instance, the cellular component is a lipid or a protein.

In a particular embodiment, the length of the nucleic acid fragment isolated in the cell free complexes is over 500, over 750, over 1000, over 2000 or over 50000 base pairs. In another embodiment, the lent hog the nucleic acid fragments in the cell free complex is between 500 and 5000 base pairs, 1000 to 10,000 base pairs, 1000 to 5000 base pairs, or 10,000 to 20,000 base pairs.

In a particular embodiment of the foregoing, the nucleic acid is DNA, RNA or a combination of the foregoing. In a further particular embodiment, the nucleic acid is DNA. In a further particular embodiment, the nucleic acid is RNA.

In a particular embodiment of the foregoing, the associated cellular component is a lipid, a protein, a polypeptide or a combination of the foregoing. In a further particular embodiment, the cellular component is a lipid. In a further particular embodiment, the cellular component is a protein. In a further particular embodiment, the cellular component is a polypeptide.

In a particular embodiment of the foregoing, the circulating cell free nucleic acid may be subject to additional processing steps, for example, to further isolate the cell-free nucleic acid from a cell free complex or associated cellular components. Any means known in the art to accomplish such ends is included in the scope of the present disclosure.

### Biologic Profiles

The present disclosure also allows the generation of biologically relevant profiles of the cell-free nucleic acid, polypeptides and/or lipids isolated as described herein. In one embodiment, the profile comprises one or more nucleic acid, polypeptide or lipid characteristics. In another embodiment, the profile comprises one or more nucleic acids, polypeptides or lipids isolated as described herein. Such biological profiles may be used for various purposes, including but not limited to, monitoring the health of a subject over time and for use in methods of diagnosis or treatment.

In one embodiment, the present disclosure provides a method for determining a biologically relevant nucleic acid, polypeptide or lipid profile of a subject, the method comprising isolating a cell free complex comprising circulating cell free nucleic acid and associated cellular components, optionally further processing the complex and identifying one or more nucleic acids, polypeptides and/or lipids present in the complex. Such method may further comprise comparing the subject profile to a corresponding profile indicative of a disease state and determining the subject is suffering from or at risk for a particular disease or condition if the subject profile contains one or more characteristics of the corresponding profile indicative of a disease state. Such corresponding profile may be a disease fingerprint as described herein.

In one embodiment, the present disclosure provides a method for determining a biologically relevant nucleic acid, polypeptide or lipid profile of a subject, the method comprising isolating a cell free complex comprising circulating cell free nucleic acid and associated cellular components, optionally further processing the complex and identifying one or more nucleic acid, polypeptide or lipid characteristics of the nucleic acids, polypeptides and/or lipids present in the complex. Such method may further comprise comparing the subject profile to a corresponding profile indicative of a disease state and determining the subject is suffering from or at risk for a particular disease or condition if the subject profile contains one or more characteristics of the corresponding profile indicative of a disease state. Such corresponding profile may be a disease fingerprint as described herein.

In one embodiment, the present disclosure provides a method for determining a biologically relevant nucleic acid profile of a subject, the method comprising isolating a cell free complex comprising circulating, cell-free nucleic acid and associated cellular components as set forth in any one the methods described herein, optionally further processing the cell free complex and identifying a plurality of cell-free nucleic acids or one or more nucleic acid characteristics to produce the nucleic acid profile. Such method may further comprise comparing the subject profile to a corresponding nucleic acid profile indicative of a disease state and determining the subject is suffering from or at risk for a particular disease or condition if the subject profile contains one or more characteristics of the corresponding nucleic acid profile indicative of a disease state. Such corresponding profile may be a disease fingerprint as described herein.

In one embodiment, the present disclosure provides a method for determining a biologically relevant polypeptide profile of a subject, the method comprising isolating a cell free complex comprising circulating, cell-free nucleic acid and associated cellular components as set forth in any one the methods described herein, optionally further processing the cell free complex and identifying a plurality of polypeptides or one or more polypeptide characteristics to produce the polypeptide profile. Such method may further comprise comparing the subject profile to a corresponding polypeptide profile indicative of a disease state and determining the subject is suffering from or at risk for a particular disease or condition if the subject profile contains one or more characteristics of the corresponding polypeptide profile indicative of a disease state. Such corresponding profile may be a disease fingerprint as described herein.

In one embodiment, the present disclosure provides a method for determining a biologically relevant lipid profile of a subject, the method comprising isolating a cell free complex comprising circulating, cell-free nucleic acid and associated cellular components as set forth in any one the methods described herein, optionally further processing the cell free complex and identifying a of lipids or one or more lipid characteristics to produce the lipid profile. Such method may further comprise comparing the subject profile to a corresponding lipid profile indicative of a disease state and determining the subject is suffering from or at risk for a particular disease or condition if the subject profile contains one or more characteristics of the corresponding lipid profile indicative of a disease state. Such corresponding profile may be a disease fingerprint as described herein.

In one embodiment, the present disclosure provides a method for determining a biologically relevant nucleic acid profile of a subject, the method comprising isolating circulating, cell-free nucleic acid as set forth in any one of the nineteenth to twenty-second aspects, optionally further processing the circulating, cell-free nucleic acid and identifying a plurality of nucleic acids to produce the nucleic acid profile. Such method may further comprise comparing the subject profile to a corresponding nucleic acid profile indicative of a disease state and determining the subject is suffering from or at risk for a particular disease or condition if the subject profile contains one or more characteristics of the corresponding nucleic acid profile indicative of a disease state. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA. Such corresponding profile may be a disease fingerprint as described herein.

In another embodiment, the present disclosure provides for a method of determining a disease fingerprint of a particular disease or condition, the method comprising isolating a cell free complex comprising circulating cell free nucleic acid from a subject known to have a particular disease or condition (or known/deemed to be healthy or not suffering from the disease or condition), optionally further processing the cell free complex, generating a nucleic acid, polypeptide or lipid profile of the disease or condition, generating a corresponding nucleic acid, polypeptide or lipid profile from a subject that is not suffering from a particular disease or condition, and comparing the disease and control profiles to identify nucleic acid, polypeptide and/or lipid characteristics in the disease profile that are absent in the control profile or that are present in the control profile, but absent in the disease profile. Such characteristics may be referred to as a disease fingerprint and compared to the corresponding profiles generated from subjects according to the methods of the present disclosure

### Methods of Diagnosis

Through the use of the isolated cell free complexes of the present disclosure, it can be determined if a subject is suffering from or at risk for a disease or condition. The disease or condition may be any disease or condition known in the art, such as for example, cancer, heart disease, or diseases and conditions associated with genetic or protein abnormalities.

In one embodiment, the present disclosure provides a method for determining if a subject is suffering from or at risk for a disease, the method comprising isolating a cell free complex comprising circulating, cell-free nucleic acid and associated cellular components or circulating cell-free nucleic acid as set forth in any one of the methods described herein, optionally further processing the cell free complex and determining the presence of a characteristic associated with the disease in the components of the complex. Such characteristic may be the presence of nucleic acid characteristic, a proteomic characteristic or a lipid characteristic.

In another embodiment, the present disclosure provides a method for determining if a subject is suffering from or at risk for a disease, the method comprising isolating a cell free complex comprising circulating, cell-free nucleic acid and associated cellular components or circulating cell-free nucleic acid as set forth in any one of the methods described herein, optionally further processing the cell free complex and determining the presence of a nucleic acid characteristic in the cell-free nucleic acid. In one embodiment the subject is judged to be suffering from or at risk for a disease if the presence of any of the characteristic is associated with the disease. Such nucleic acid characteristic includes, but is not limited to, a mutation, a polymorphism, methylation state, concentration of a nucleic acid, level of expression of a nucleic acid, the profile of the nucleic acids or a combination of the foregoing.

In another embodiment, the present disclosure provides a method for determining if a subject is suffering from or at risk for a disease, the method comprising isolating a cell free complex comprising circulating, cell-free nucleic acid and associated cellular components as set forth in any of the methods described herein, optionally further processing the cell free complex and determining the presence of a polypeptide and/or lipid characteristic in the cellular component. In one embodiment, the subject is judged to be suffering from or at risk for a disease if the presence of any of the characteristic is associated with the disease. Such polypeptide characteristic includes, but is not limited to, a mutation, the presence of post-translational modifications, the presence of insertions or deletions, the concentration, level of expression of a nucleic acid, the profile of the polypeptides or a combination of the foregoing. Such lipid characteristic includes, but is not limited to, the presence of altered forms, the presence of modifications, the concentration, the expression level, the profile of lipids or a combination of the foregoing.

In another embodiment, the present disclosure provides a method for determining if a subject is suffering from or at risk for a disease, the method comprising generating a nucleic acid, polypeptide or lipid profile from a subject and comparing the generated profile to a corresponding disease fingerprint and determining the subject is suffering from or at risk for a particular disease or condition if the subject profile contains one or more characteristics of the disease fingerprint.

In another embodiment, the present disclosure provides a method for determining if a subject is suffering from or at risk for a disease, the method comprising isolating circulating, cell-free nucleic acid as set forth in any one of methods described herein, optionally further processing the cell-free nucleic acid and determining the presence of a nucleic acid characteristic. In one embodiment, the subject is judged to be suffering from or at risk for a disease if the presence of any of the characteristic is associated with the disease. Such nucleic acid characteristic includes, but is not limited to, a mutation, a polymorphism, methylation state, concentration of a nucleic acid, level of expression of a nucleic acid, the profile of the nucleic acids or a combination of the foregoing. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA.

In another embodiment, the present disclosure provides a method for determining if a subject is suffering from or at risk for a disease, the method comprising generating a nucleic acid profile from a subject as set forth in any of the methods described herein and comparing the generated profile to a corresponding disease fingerprint and determining the subject is suffering from or at risk for a particular disease if the subject profile contains one or more characteristics of the disease fingerprint. In one embodiment of this aspect, the circulating, cell-free nucleic acid is present in a cell free complex and such method may further comprise isolating at least one cell-free nucleic acid from the complex. In another embodiment, the circulating, cell-free nucleic acid is associated with a cellular component, such as, but not limited to, a polypeptide and/or a lipid, and such method may further comprise isolating at least one cell-free nucleic acid from the cellular components. Such method may further comprise isolating at least one cellular component from the circulating, cell-free nucleic acid and analyzing the cellular component. In one instance, the cellular component is a lipid or a protein. Such nucleic acid may be DNA and/or RNA

In any of the foregoing, the circulating, cell-free nucleic acid and cell free complex may be isolated by any method described herein. The determined characteristic, whether a nucleic acid, polypeptide or lipid characteristic, may inform a healthcare provider if the subject is suffering from or at risk for a particular disease or condition. For example, if a nucleic acid characteristic is analyzed, such as the presence of a mutation disposing an individual to a cancer risk, the individual may be monitored for the increased risk, undergo additional testing or initiate lifestyle changes or a combination of the foregoing.

Furthermore, in the foregoing embodiment, the determined characteristic, whether a nucleic acid, polypeptide or lipid characteristic may be compared to a control to determine if the subject is at risk for a particular disease or condition. For example, a nucleic acid profile of the nucleic acid components in the cell free complex may be prepared as described herein. The nucleic acid profile may be compared to a control nucleic acid profile obtained under the same or similar conditions that is free of a particular disease. Furthermore, profiles may be taken from individuals known to have a particular disease or condition, and these profiles compared to a control profile to identify nucleic acids that are subject to increased or decreased concentration to generate a nucleic acid fingerprint of a particular disease or condition comprising such nucleic acids that undergo changes in concentration. The presence of one or more such nucleic acids in a subject nucleic acid profile may indicate that the subject is suffering from or at risk for the disease or condition. The same analysis may be undertaken for the polypeptide components and the lipid components of the cell free complexes.

### Determining Methods of Treatment

The present disclosure also provides for determining a beneficial or optimal course of treatment for an individual. Such treatment decisions can be of use to a healthcare provider when choosing between various forms of treatment, especially when physiological feature of the subject may impact the efficacy of such treatment or inform the healthcare provider on the dose of a particular medication to be used in such treatment.

In one embodiment, the present disclosure provides a method for determining beneficial or optimal course of treatment, the method comprising isolating a cell free complex comprising circulating cell free nucleic acid and associated cellular components or circulating cell-free nucleic acid, optionally further processing the cell free complex and determining the presence of a characteristic that may impact a particular course of treatment and modifying the course of treatment if the characteristic is present. Such characteristic may be the presence of genomic characteristic, a proteomic characteristic or a lipidomic characteristic.

For example, consider planning a course of treatment using the drug Plavix. Plavix is used to prevent blood clots after a recent heart attack or stroke, and in people with certain disorders of the heart or blood vessels. The effectiveness of Plavix in some individuals is dependent on the metabolism of the drug by certain liver enzymes, particularly CYP2C19. Metabolism is required for optimal effectiveness of the drug. Certain polymorphisms are indicative of those individuals that do not metabolize Plavix effectively and as a result may not receive the full benefits of the drug at standard dosing. By identifying such polymorphisms, a healthcare provider may plan the most beneficial or optimal course of treatment by prescribing another drug or adjusting the administered dose of Plavix.

### Cell Free Complexes

The present disclosure provides methods for the isolation of cell free complexes comprising a cell free nucleic acid component and at least one cellular component. The at least one cellular component may be associated with a nucleic acid directly, or indirectly (such as through interaction with another cellular component). Such additional cellular components include but are not limited to, polypeptides, proteins and lipids. The presence of the at least one cellular component may aid in the preservation of the nucleic acid component, such as by protecting the nucleic acid component from degradation. As a result, nucleic acids of increased molecular weight are isolated by the methods of the present disclosure as compared to prior art methods. In one embodiment, at least 50%, 60%, 70%, 80%, 90%, 95% or 99% of the nucleic acids in the cell free complex are associated with at least one additional cellular component.

The cell free complex isolated as disclosed herein may be analyzed as is known by one of ordinary skill in the art. For example, the nucleic acid component may be analyzed by any known genomic methods to determine a genomic characteristic, the polypeptide component may be analyzed by any known proteomic methods to determine a proteomic characteristic and the lipid component may be analyzed by any known lipidomic methods to determine a lipidomic characteristic. As such, information regarding the status of the subject may be obtained and used for diagnostic, prognostic or therapeutic applications.

### Samples

In one embodiment of the above method, a sample is obtained from the patient. Any type of sample routinely used in the art may be used, such as but not limited to, blood, plasma, serum, bone marrow, urine, amniotic fluid, placental blood, buccal swabs or other sample types. In one embodiment, the sample is a blood sample or plasma obtained from the patient. The blood sample may be collected and processed as is known in the art. In one embodiment, the blood samples are collected in an anti-chelating agent such as EDTA or another calcium binding agent. The blood sample may be subject to purification, such as, but not limited to, centrifugation, in order to separate the plasma fraction from other components of the blood. In one embodiment, the sample is a plasma sample. The sample may be stored at 4 degrees Celsius or lower prior to the purification step if desired. The sample may be stored cryogenically after purification for future analysis. In one embodiment, the sample is may be processed to remove cellular components, such as blood cells. In one embodiment, the sample is not subject to steps to purify or concentrate nucleic acid or another cellular component contained in the sample, with the understanding that procedures to separate plasma from blood and similar processing steps are not considered to be steps that purify or concentration nucleic acid or another cellular component contained in the sample.

### Isolation of Cell Free Complexes and Circulating Cell-Free Nucleic Acid

The circulating cell-free nucleic acid and cell free complexes are separated using centrifugation. The embodiments described herein are useful in isolating both circulating cell-free nucleic acid and cell free complexes. In certain embodiments, the methods isolate both circulating cell-free nucleic acid and cell free complexes. In one embodiment, density gradient centrifugation is used. In a particular embodiment vertical spin density gradient ultracentrifugation is used. However, any density gradient separation means known in the art may be used.

In a typical samples, using density gradient centrifugation, the circulating cell-free nucleic acid and/or cell free complexes are separated from other components in the sample. For a typical sample containing free nucleic acid, nucleic acid complexes and polypeptides/proteins, the foregoing are separated in the following order (from the bottom of the density gradient to the top of the density gradient): polypeptide/proteins, circulating cell-free nucleic acid (which may be associated with cellular components) and cell free complexes as described herein and free nucleic acid. A variety of density gradient centrifugation conditions may be used. The composition of the density gradient may impact the separation between various components in the sample. The following are illustrated by way of example only and should not be interpreted as limiting the scope of the separation techniques to density gradient centrifugation or as limiting the conditions employed in density gradient centrifugation to those conditions specified.

As discussed above, in some cases certain a given sample may contain more than one type of component. Specific density gradient centrifugation conditions may be employed to provide maximum resolution of the various components in a sample or may be employed to provide maximum resolution of a selected component in the sample, such as the cell free complexes described herein.

In one embodiment, the density gradient centrifugation conditions and parameters are selected to provide maximum resolution of the circulating cell-free nucleic acid and/or cell free complexes described herein. Conditions and parameters that may be varied include, but are not limited to, density of the gradient, density of the layers comprising the density gradient, volume of the layers comprising the density gradient, centrifugation time settings, acceleration setting (impacting the time it takes for the centrifuge to reach a set RPM), deceleration settings (impacting the time it takes for the centrifuge to come to a stop from the set RPM at the end a specified time setting), speed of the centrifuge (measured in RMP), centrifugal force applied to the sample (measured in x g) and temperature of the centrifugation run. The various parameters discussed above may be varied singly or in any combination desired.

In one embodiment, the density gradient comprises a formed gradient of uniform composition. In another embodiment, the density gradient comprises two layers of gradient material (referred to as a top and bottom layer). A commonly used density gradient material is CsCl. Other commonly used density gradient materials include KBr, sucrose, and colloidal silica particles coated with polyvinylpyrrolidone (such as the product sold as Percoll®). Any density gradient solution known in the art to create the required density range may be used. In one embodiment, the density gradient is formed from CsCl. Centrifugation will be performed in an appropriate vessel, such as a centrifuge tube. A variety of suitable centrifuge tubes are commercially available, for example from Beckman-Coulter, of Brea, Calif. In a specific embodiment separation is achieved using a single spin.

In one embodiment, the density of the gradient is from 1.30 to 1.45 g/cm³. In another embodiment, the density of the gradient is from 1.30 to 1.40 g/cm³. In another embodiment, the density of the gradient is from 1.35 to 1.40 g/cm³. In another embodiment, the density of the gradient is from 1.35 to 1.45 g/cm³. In another embodiment, the density of the gradient is from 1.38 to 1.43 g/cm³. In another embodiment, the density of the gradient is from 1.39 to 1.42 g/cm³. In another embodiment, the density of the gradient is from 1.35 to 1.42 g/cm³. In one embodiment, the volume of the density gradient is from 3-5 mls. In one embodiment, the volume of the density gradient is 5.0 mls.

In one embodiment where two layers of density gradient material are used, the bottom layer ranges from 1.10 to 1.40 g/cm³, from 1.15 to 1.30 g/cm³ or from 1.15 to 1.25 g/cm³ and the density of the top layer ranges from 0.5 to 1.2 g/cm³, from 1.0 to 1.15 g/cm³ or from 1.0 to 1.10 g/cm³. In another aspect of this embodiment, the density of the bottom layer is 1.21 g/ml or 1.30 g/cm³ and the density of the top layer is 1.05 g/cm³. Further, in one aspect of this embodiment, the volume of the bottom layer ranges from 0.2 to 4.0 ml, from 0.8 to 2.5 ml or from 1 to 2 ml and the volume of the top layer ranges from 1 to 4.8 ml, from 1.2 ml to 3.0 ml or from 3.0 to 4.0 ml. In another aspect of this embodiment, the volume of the bottom layer is 2.0 ml or 1.0 ml and the volume of the top layer is 2.90 ml or 3.9 ml.

Further, in one aspect of this embodiment, the settings for the ultracentrifuge are varied as follow: (i) centrifugation time from 1 to 4 hours (note that centrifugation time does not include the time required for deceleration of the centrifuge rotor), from 1 to 3 hours or from 2 to 4 hours; (ii) centrifugation speed from 190,000×g to 555,000×g or 275,000×g to 480,000×g; and (iii) centrifugation temperature from 15 to 30 degrees Celsius or from 20 to 25 degrees Celsius. Furthermore, in one aspect of this embodiment, the acceleration and deceleration settings are selected provide appropriate acceleration and deceleration profiles in order to maximize the desired separation. In one aspect, the acceleration and/or deceleration phases of the spin are set to be slow in order to minimize vibrations that may occur during a quick acceleration and/or deceleration. In one aspect, the acceleration and/or deceleration phases of the spin are set to be fast in order to resolve a given class of lipoprotein. For example, using a Beckman Coulter ultracentrifuge (Optima™ XL-100 K Ultracentrifuge), the acceleration and/or deceleration settings may range from 5 to 9 or 8 to 9 (with 9 being the slowest setting). In another aspect of this embodiment, the acceleration and/or deceleration settings may range from 1 to 5 or 2 to 4 (with 1 being the fastest setting).

In one embodiment, the following conditions are used: (i) density gradient of 1.30 to 1.45 g/cm3; (ii) 3 hour spin time; (iii) 20 degrees Celsius; (iv) 416,000×g and (v) minimum acceleration/deceleration settings. TIMES G

### Use of Comparative Database

As disclosed herein, the results of analysis of the components of the cell free complex, including cell-free nucleic acid, obtained as described herein may be compared with a standard or control. For example, such a control may be a sample from a subject who is determined to have or be free of a given disease or condition. Furthermore, the control may be a compilation of the results obtained from a population of individuals who are determined to have or be free of a given disease or condition. In such a case, the results may be contained within a comparative database.

When a comparative database is used as a control, the comparative database may be constructed in a variety of ways. Furthermore, the individuals in the comparative database may be matched to the subject being tested based on a stratification criterion or may be non-matched as compared to the subject. In one embodiment, the stratification criterion is age. In another embodiment, the stratification criterion is ethnic origin. For example, if the subject is 65 years of age, in one embodiment the comparative database may be composed of individuals with ages from, for example, 60 to 70 years, or in a second embodiment, the comparative database may be composed of individuals with ages from, for example, 25 to 40 years. The use of a comparative database comprising a younger population may offer certain advantages since the younger subjects that comprise the population will be more likely to be free of disease states and other conditions that may impact the analysis. Using an age matched population for the comparison may actually decrease the sensitivity of the method since the age matched population of the comparative database may in fact have a certain risk for the disease or condition being analyzed.

The individuals making up the comparative database may be healthy (i.e., disease free) or they may be selected based on their diagnosis of a particular disease or condition, or both. If healthy individuals are selected, the characteristic determined from the subject, such as the presence of a particular mutation or polymorphism or the nucleic acid and/or polypeptide profile, can be compared with the corresponding characteristic from healthy individuals in the comparative database. If individuals with a diagnosed disease state are selected, the characteristic determined from the subject, such as the presence of a particular mutation or polymorphism or the nucleic acid and/or polypeptide profile, can be compared with the corresponding characteristic from individuals diagnosed with a disease states and/or defined stages of a disease state in the comparative database. In this manner, the comparison may be able to predict if a subject is at risk for a particular disease or condition (from a comparison with healthy individuals in the comparative database), if the subject is suffering from a particular disease or condition (from a comparison with individuals in the comparative database diagnosed with such disease or condition) or to diagnose severity (from a comparison with individuals in the comparative database diagnosed with various stages of a disease or condition). The stratification of the database, as discussed below, may aid in making such comparisons.

The comparative database may be stratified based on a number of stratification criteria. These criteria may be risk factors, demographic factors, other relevant factors or a combination of the preceding. Demographic factors include, but are not limited to, age, gender and ethnicity. The inclusion of a specific stratification criteria as a risk factor or demographic factor may be modified (for example, age may be considered both a risk factor and a demographic factor). The individuals in the comparative database may be tagged or otherwise identified, such that the appropriate population of individuals in the comparative database may be selected for the comparison to the subject.

Furthermore, the comparative database may be refined over time. The individuals in the database may be followed over time and their health status monitored. If an individual no longer meets an inclusion criterion for the comparative database, the individual may be removed or their information modified. In this manner the quality of the comparative database may be improved over time, resulting in a database with improved sensitivity and specificity.

The characteristic determined from the subject, such as the presence of a particular mutation or polymorphism or the nucleic acid, lipid and/or polypeptide profile, may then be compared to the corresponding characteristic from appropriately selected defined group of individuals in a comparative database. Appropriately selected means that the selected characteristic from a defined group of individuals in the comparative database is selected for comparison to the selected characteristic determined for the subject. The defined group may be all the individuals in the comparative database or less than all the individuals in the comparative database. The defined group may be selected on the basis of stratification criteria as discussed above. The healthcare provider may select the defined group, with such selection based on one or more defining characteristics of the subject. In one embodiment, the defined group may be selected on the basis of ethnicity (African-American), gender (male), health status (disease free or diagnosed with a particular disease or condition), and age (20-45 years of age or 55-65 years of age). Furthermore, the comparison may be carried out multiple times for any given subject to various iterations of the comparative database. For example, given a 60 year old, non-smoking, African-American male subject, comparisons could be made using a defined group from the database selected on the basis of gender (male) only, gender and age, or selected to include all individuals in the comparative database.

### Results

### Example 1—General Procedures

### Sample Handling

Blood samples were collected in 10 ml lavender top EDTA collection tubes using standard phlebotomy practices. Immediately after collection, the tubes were gently inverted 4-6 times and then centrifuged at 2,500 RPM for 15 minutes at 4° C. The supernatant plasma was transferred into 2 ml cryogenic vials and frozen at −80° C. until analysis. No further modification of manipulation of the blood sample is required. The supernatant plasma is used directly in the methods as described without further amplification of purification of any component of the plasma, such as, but not limited to, nucleic acids and polypeptides.

### VAP Analysis

VAP cholesterol patent tests were performed at Atherotech Diagnostic Laboratories (Birmingham, Ala.) according to internal standard operating procedures. The VAP method separates lipoproteins based on their density using single vertical-spin density gradient ultracentrifugation, then quantifies cholesterol content (Kulkarni, et al., Clin Lab Methods, 26, 787-802, 2006; Kulkarni et al., J. Lipid Res, 35, 159-168, 1994; Kulkarni et al., J Lipid Res, 38, 2353, 2364, 1997).

### Density Gradient Centrifugation

Twenty μl of plasma was diluted in 980 μl of 1× phosphate buffered saline (PBS) prior to mixing with a CsCl gradient solution. The CsCl gradient was prepared by adding 2.19 g of CsCl to 4 ml of 1× Tris-EDTA buffer; sample volume was adjusted to 5 ml to a final density of 1.30 to 1.45 g/cm3. The resulting sample was centrifuged using a Beckman Coulter Optima XL-100K ultracentrifuge and Beckman VTI 65.2 rotor 3 hours at 65,000 RPM at 20° C. (416,000×g). 250 μl fractions (fractions 1-20) were collected with a peristalitic pump. Detection and quantification of the cell free nucleic acid was conducted by measuring uv absorbance at 260 nm by a NanoDrop ND-8000 spectrophotometer (ThermoScientific).

For further analysis of the nucleic acid, polypeptide and lipid components, the fractions were desalted and concentrated on a Microcon DNA fast flow centrifugal filter column. Nucleoprotein complexes in the solution were disassociated by alkaline denaturation. Free nucleic acid complexes were subject to isothermal amplification to amplify cell free DNA acid or reverse transcription on cell free RNA. Subsequent analysis was carried out using commercially available kits according to manufacturers' instructions.

### Example 2—Overview of General Procedure

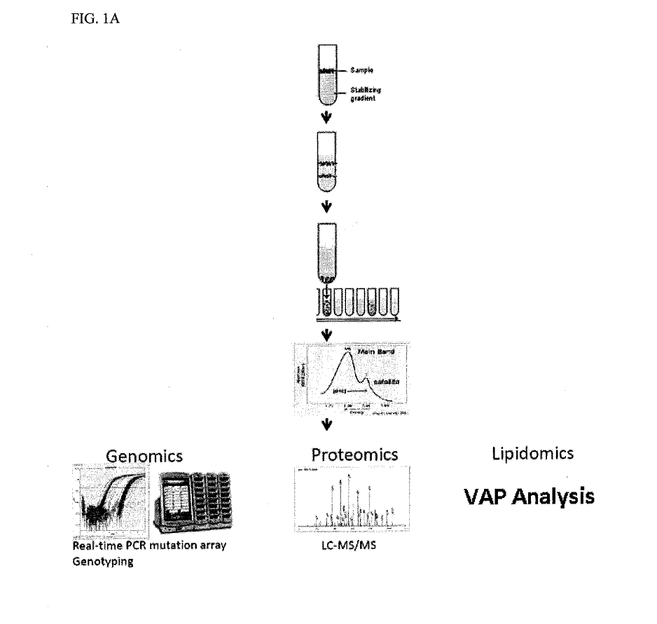

FIG. 1 shows an overview of one embodiment of the general procedure of the methods as disclosed herein. As shown in FIG. 1, a sample (in this example, a blood sample processed as described above). The sample is added to an appropriate vessel for density gradient centrifugation. Any suitable vessel may be used as is known in the art. In this example, a Beckman Coulter OptiSeal tube is used. The sample is then subject to density gradient centrifugation as described herein. While a number of density gradient centrifugation procedures may be used as is known in the art (including those described herein), in many of the examples described herein the density gradient is a CsCl density gradient to yield a final density of 1.3 to 1.45 g/cm³. The centrifugation protocols may also be varied as is known in the art (including those protocols described herein. In many of the examples described herein, the centrifugation conditions are as follow: 3 hour spin at 65,000 rpm at 20° C. using a Beckman Coulter Optima XL-100K ultracentrifuge and Beckman VTI 65.2 rotor (416,000×g).

At the end of the centrifugation procedure, the vessel containing the sample is gently removed and the bottom of the tube is punctured to allow removel of the sample from the bottom of the tube. Fractions are collected using a peristaltic pump for analysis. Using this method, the bulk nucleic acid (containing less or no associated cellular components) is lower in the density gradient and is collected first (generally having a density around 1.70 g/cm³), followed by the cell free nucleic acid associated with cellular components and cell free complexes (generally having a density around 1.30 to 1.45 g/cm³), followed by a protein fraction (generally having a density around 1.25 g/cm³). Any convenient aliquot volume may be used, for example 250 μl fractions. The collected fractions are subject to ultraviolet absorbance measurement at 260 nm using a spectrophotometer (such as a NanoDrop ND-8000) to detect and quantify the nucleic acid and other components present in the collected fractions.

The fractions may then be further processed as is known in the art for subsequent analysis. For example, for genomic analysis the fractions may be deslated and concentrated using methods known in the art (for example, a Microcon DNA Fast Flow centrifugal filter column). Nucleoprotein complexes may be dissociated using standard techniques (such as alkaline denaturation). Other processing steps may also be performed as is known in the art. In one embodiment, such further processing steps do not involve the purification of nucleic acid isolated. Subsequent analytic techniques include, but are not limited to genomics, proteomics and lipidomics. Genomic analysis includes, but is not limited to, gene profiling and identification, analysis of methylation patterns, mutation analysis, polymorphism analysis and gene expression analysis. Proteomic analysis includes, but is not limited to, polypeptide profiling and identification, mutation analysis, polypeptide modification analysis and polypeptide expression analysis. Lipidomic analysis includes, but is not limited to, lipid profiling and identification, lipid modification analysis and lipid expression analysis.

### Example 3—Characterization of Isolated Fractions

FIG. 1B shows a representative chromatogram and quantitative peak analysis of the nucleic acid isolated by the methods described herein. FIG. 1B shows 2 separate samples, designated VAP3 (leftmost graph) and VAP4 (rightmost graph). As can be seen in FIG. 1B, the samples generate a major peak and a minor peak; the major peak corresponds to fraction 13 collected from the density gradient as described below and contains all or majority of the circulating cell-free nucleic acid and cell free complex. Table 1 shows the quantitative measurements for each histogram. As shown in Table 1, the various characteristics of the histograms can be quantitated using the variables described; other variables may also be used as is known in the art. From these variables, an index factor can be generated for each sample to characterize each sample and allowing the ultimate differentiation of healthy subjects and subjects that may be suffering from or pre-disposed to a particular disease or condition. Such an index factor may be generated by using the variables shown in Table 1 or other variables known in the art. For example, the index factor may be calculated by subtracting the height of the major and minor peaks and dividing this difference by the difference of the retention time of the major and minor peaks. Using the values in Table 1 for the VAP3 sample, the calculation would be (372.65−75.01)/(18.03−20.41). Alternatively, another variable, such as the width of the major and minor peaks may be used. Using the values in Table 1 for the VAP3 sample, the calculation would be (257.6−122.6)/(18.03−20.41). The above are exemplary only and other variables or combinations of variables may be used to characterize the samples. In addition, specific properties of the circulating cell-free nucleic acid and/or the cell free complex or the components thereof, such as the presence of one or more mutations, polymorphisms, post-translational modifications and the like may also be used, either alone or in combination with the characteristics described above, to characterize a sample.

For FIG. 1B, samples were prepared as described in Example 1. Conditions for density gradient preparation and centrifugation conditions were: CsCl density gradient providing a final density of 1.32 to 1.40 g/cm³; 3 hour spin at 65,0000 rpm at 20° C. using a Beckman Coulter Optima XL-100K ultracentrifuge and Beckman VTI 65.2 rotor (416,000×g). Fractions (250 ul) were collected from the bottom of the centrifuge vessel using a peristaltic pump; 20 fractions were collected. Under these conditions, fraction 13 contains the circulating cell-free nucleic acid/cell free complex comprising circulating, cell free nucleic acid bound to cellular components.

| | Ret | | | | High | | Width | | Asym | Trailing |
|---|---|---|---|---|---|---|---|---|---|---|
| Sample | Time | Area | Area % | Height | Value | Width | 50% | Res | 10% | 10% |
| VAP3 | 18.03 | 35525.77 | 14.93 | 372.65 | 367.78 | 257.6 | 80.8 | 0.876 | 1.6 | 1.3 |
| | 20.41 | 3188.21 | 1.34 | 75.01 | 70.28 | 122.6 | 245.2 | 0.744 | 0.1 | 0.55 |
| VAP4 | 26.67 | 40841.19 | 17.16 | 362.62 | 357.85 | 275 | 129.4 | 0.507 | 0.65 | 0.82 |
| | 27.94 | 6616.56 | 2.78 | 204.92 | 200.09 | 165.2 | 164.8 | 2.827 | 0.12 | 0.56 |

As discussed herein, the methods described separate circulating cell-free nucleic acid/cell free complex comprising a pool of circulating cell free nucleic acid that are associated with cellular components, such as polypeptides and lipids, from circulating cell free nucleic acid that is not associated with such cellular components (or not associated with such cellular components to an appreciable degree) and polypeptides. Each of these classes migrates in a distinct pattern during density gradient centrifugation. As described herein, the circulating cell free nucleic acid that is associated with a cellular component represents a biologically relevant fraction of such nucleic acids and as a result, the methods described herein provide a mechanism to isolate this biologically relevant pool of circulating, cell free nucleic acid for analysis.

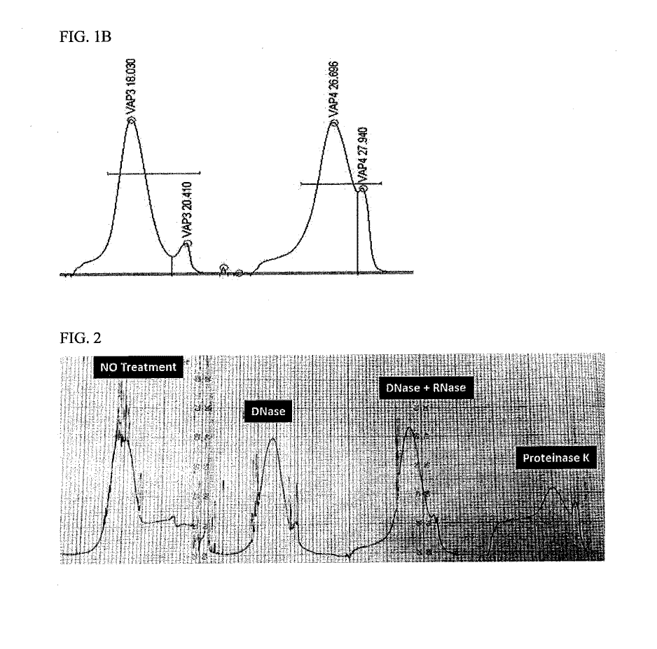

In order to further characterize the material isolated, the plasma samples were prepared as described above and treated with various agents before being subject to density gradient centrifugation (conditions for sample preparation, density gradient preparation and centrifugation were as described for FIG. 1B). Chromatograms were generated by monitoring ultraviolet absorbance at 260 nm (detecting nucleic acids and polypeptides). Samples were treated with DNase I (1-1000 μg/ml for 1 hr at 37° C.) to non-specifically cleave double stranded DNA, DNase (1-1000 μg/ml for 1 hr at 37° C.) and RNase A (1-1000 μg/ml for 1 hr at 37° C.) to non-specifically cleave RNA and proteinase K (50-250 μg/ml for 1-4 hrs at 56° C.) to non-specifically cleave polypeptides. Reactions with DNase, RNase and proteinase K were carried out according to manufacturers' instructions. As shown in FIG. 2, the leftmost chromatogram illustrates no treatment, followed by DNase I treatment, DNase I and RNase A treatment, and proteinase K treatment (rightmost chromatogram). As can be seen treatment with each agent modified the resulting chromatogram indicating that each of DNA, RNA and nucleic acid/polypeptide complexes polypeptides are present in the material isolated by the methods described herein.

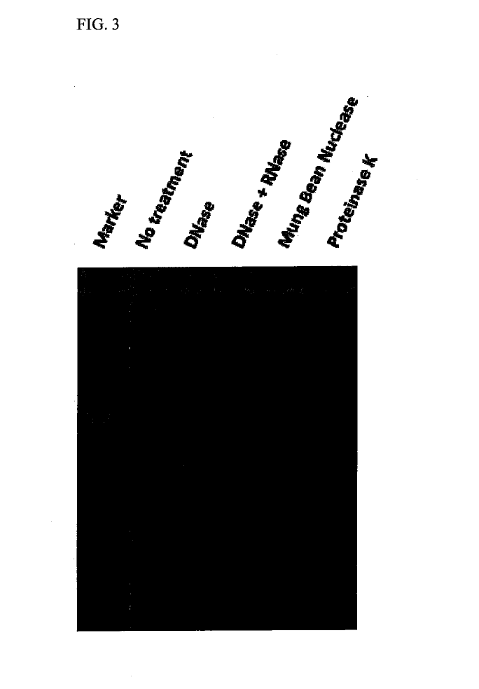

In FIG. 3, the conditions for sample preparation, density gradient preparation and centrifugation were as described for FIG. 1B. In this FIG. the peak fraction for each sample (fraction 13) were collected and desalted to remove CsCl salt using a Microcon DNA Fast Flow Centrifugal Filter Unit and re-suspended in 25 μl of water. Samples re-suspended in water were treated with DNase I (1-1000 μg/ml for 1 hr at 37° C.) to non-specifically cleave double stranded DNA, DNase I (1-1000 μg/ml for 1 hr at 37° C.) and RNase A (1-1000 μg/ml for 1 hr at 37° C.) to non-specifically cleave both DNA and RNA, mung bean nuclease (1-5 U/ug DNA) to cleave single stranded nucleic acid and proteinase K (50-250 μg/ml for 1-4 hrs at 56° C.) to non-specifically cleave polypeptides. The treated samples were subject to agarose gel electrophoresis according to standard procedures. Reactions with DNase I, RNase A, mung bean nuclease and proteinase K were carried out according to manufacturers' instructions. The gel in FIG. 3 shows molecular weight markers in lane 1, control (no treatment) sample in lane 2, sample treated with DNase I in lane 3, sample treated with DNase I and RNase A in lane 4, sample treated with mung bean nuclease in lane 5 and sample treated with proteinase K in lane 6. Agarose gel was run under standard conditions and stained with ethidium bromide. As shown in FIG. 3, treatment with each agent modified the resulting chromatogram indicating that each of double and single stranded DNA, RNA and nucleic acid/polypeptide complexes are present in the material isolated by the methods described herein.

### Example 4—Comparison of the Methods of the Present Disclosure with Commercially Available Extraction Kits

Table 2 shows a comparison of the methods of the present disclosure with two commercially available kits for the isolation of circulating cell free nucleic acid. Procedures for the commercially available kits were carried out according to manufacturers' instructions. The commercially available kits were the NucleoSpin Kit from Clontech and the QIAamp Circulating Nucleic Acid Kit from Qiagen.

The QIAamp Circulating Nucleic Acid Kit simplifies isolation of circulating DNA and RNA from plasma or serum. No phenol-chloroform extraction is required and nucleic acids bind specifically to the QIAamp Mini column, while contaminants pass through. PCR inhibitors, such as divalent cations and proteins, are completely removed in 3 wash steps, leaving pure nucleic acids to be eluted in a buffer provided with the kit. The QIAamp Circulating Nucleic Acid technology yields circulating DNA and RNA from human plasma, serum, or urine. Circulating RNA can be purified with DNA digestion using the RNase-Free DNase Set.

The NucleoSpin Plasma XS kit is designed to isolate fragmented DNA larger than 50 bp from human EDTA blood plasma. NucleoSpin Plasma XS allows elution in only 5-20 μl, resulting in highly concentrated DNA.

For the methods of the present disclosure, sample preparation, density gradient preparation and centrifugation were carried out as described for FIG. 1B above. As can be seen, the methods of the present disclosure provide a method that is easier to implement, requires less sample volume and provides a significant increase in yield and purity. Furthermore, the methods provided herein offer a significant throughput for sample processing.

TABLE 2

| | | QIAamp | |
|---|---|---|---|
| | | Circulating | |
| | NucleoSpin Kit | Nucleic | Present |
| | (Clontech) | Acid Kit (Qiagen) | Disclosure |
| Throughput | 30 min/6 | 2 hr/24 | 3 hr/16 |
| | samples | samples | samples |
| Sample Volume | 200 μl | 200 μl | 20 μl |
| RNA Carrier | No | Yes | No |
| Required | | | |
| Large Volume to | Yes | Yes (up to 5 ml) | No |
| Improve Yield | | | |
| Yield, range | 0.61 to 50.11 | 9.43 to 46.27 | 97.30 to 132.25 |
| (ng/μl) n = 6 | | | |
| Yield, means ± | 12.58 ± 18.68 | 18.58 ± 13.80 | 108.46 ± 11.17 |
| SD, n = 6 | | | |
| Yield, % CV, | 148.42 | 74.26 | 10.30 |
| n = 6 | | | |
| Purity (A260/ | >1.8 | >2.0 | 0.57 to 0.63 |
| 280), n = 6 | | | |

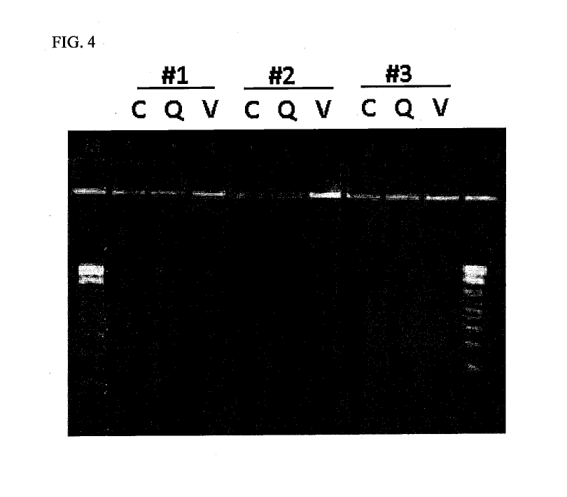

FIG. 4 shows representative agarose gels of circulating cell free DNA isolated using the methods of the present disclosure (as described in FIG. 1B) (designated V) and that prepared using the two commercially available kits described above (designated C for the NucleoSpin Kit and Q for the QIAamp kit). Fraction 13 is shown. Agarose gel was run under standard conditions and stained with ethidium bromide. In FIG. 4, three separate samples are show, with lanes M and 10 being molecular weight markers, lanes 1, 4 and 7 corresponding to DNA isolated using the NucleoSpin kit from samples 1-3, respectively, lanes 2, 5, and 8 corresponding to DNA isolated using the QIAamp kit from samples 1-3, respectively and lanes 3, 6 and 9 corresponding to DNA isolated using the methods of the present disclosure from samples 1-3, respectively. The difference in yield of DNA isolated is evident when comparing lanes 3, 6 and 9 to the remaining lanes.

### Example 5—Detection of Gene Polymorphisms from Circulating, Cell Free DNA

This example illustrates the detection of multiple gene polymorphisms on circulating cell free DNA isolated using the methods of the present disclosure as well as comparing such detection using circulating cell free DNA isolated using the commercially available kits of Example 4. For the methods of the present disclosure, circulating cell free DNA was prepared as described in for FIG. 1B, with the addition of a desalting step and disassociation of nucleoprotein complexes as described in Example 1. The commercially available kits were used according to manufacturers' instructions. Isolated DNA was used directly for genotyping experiments.

This example shows the detection of 7 single nucleotide polymorphisms (SNP) over a single gene for PCSK9, 3 SNPs over 2 genes related to warfarin sensitivity, 11 SNP's in a single gene for plavix sensitivity and 4 SNPs over 3 genes related to thrombophilia risk. The PCSK9 genotyping test was developed by the Applicants; genotyping for warfarin sensitivity, plavix sensitivity and thrombophilia risk were carried out using commercially available kits from GenmarkDx using the eSensor platform.

Table 3 shows the general PCR conditions used in the genotyping experiments while Table 4 shows the probes used in the PCSK9 genotyping experiments. Probes and primers for the commercially available kits are not available to the general public.

TABLE 3

| | | | |
|---|---|---|---|
| | | Reference | |
| | | SNP | |
| Gene Name | SNP ID | Allele | PCR Conditions |

PCSK9

| | | | |
|---|---|---|---|
| PCSK9 | rs562556 | A > G | 95° C.^{10 min} × 1 cycle; |
| PCSK9 | rs505151 | A > G | (92° C.^{15 sec}-60° C.^{1 min}) × |
| PCSK9 | rs11206510 | T > C | 50 cycles |
| PCSK9 | rs1151147 | G > T | |
| PCSK9 | rs28362286 | C > A | |
| PCSK9 | rs28362263 | G > A | |
| PCSK9 | rs7517090 | G > A | |

Thrombophilia Risk

| | | | |
|---|---|---|---|
| Factor II | 20210 | G > A | 95° C.^{4 min} × 1 cycle; |
| (Prothrombin) | | | (95° C.^{25 sec}-60° C.^{30 sec}- |
| Factor V | 1691 | G > A | 72° C.^{25 sec}) × |
| (Leiden) | | | 35 cycles |
| MTHFR | 677 | C > T | |
| | 1298 | A > C | |

Warfarin Sensitivity

| | | | |
|---|---|---|---|
| CYP450 2C19 | 430 | C > T | 95° C.^{4 min} × 1 cycle; |
| *2, *3 | 1075 | A > C | (93° C.^{45 sec}-56° C.^{45 sec}- |
| VKORC1 | −1639 | G > A | 68° C.^{45 sec}) × |
| | | | 39 cycles; |
| | | | 68° C.^{7 min} × 1 cycle |

Plavix Sensitivity

| | | | |
|---|---|---|---|
| CYP450 2C19 | 681 | G > A | 94° C.^{4 min} × 1 cycle; |
| CYP450 2C19 | 636 | G > A | (94° C.^{20 sec}-56° C.^{45 sec}- |
| CYP450 2C19 | 1 | A > G | 70° C.^{45 sec}) × |
| CYP450 2C19 | 1297 | C > T | 37 cycles; |
| CYP450 2C19 | 396 | G > A | |
| CYP450 2C19 | 19249 | T > A | |
| CYP450 2C19 | 358 | T.C | |
| CYP450 2C19 | 12784 | G > A | |
| CYP450 2C19 | 19153 | C > T | |
| CYP450 2C19 | 87290 | C > T | |
| CYP450 2C19 | −806 | C > T | |

TABLE 4

| Gene Name | SNP ID | Taqman Probe |
|---|---|---|
| PCSK9 | rs562556 | GGGGCCTACACGGATGGCCACAGCC[A/G]TCG |
| | | CCCGCTGCGCCCCAGATGAGGA |
| PCSK9 | rs505151 | AGCACTACAGGCAGCACCAGCGAAG[A/G]GG |
| | | CCGTGACAGCCGTTGCCATCTGC |
| PCSK9 | rs11206510 | AAGGATATAGGGAAAACCTTGAAAG[C/T]GA |
| | | TGTCTGTGGTGGCCGTCTTTGGC |
| PCSK9 | rs11591147 | TACGAGGAGCTGGTGCTAGCCTTGC[G/T]TTC |
| | | CGAGGAGGACGGCCTGGCCGAA |
| PCSK9 | rs28362286 | CCGTGACAGCCGTTGCCATCTGCTG[A/C]CGG |
| | | AGCCGGCACCTGGCGCAGGCCT |
| PCSK9 | rs28362263 | GGTACTGACCCCCAACCTGGTGGCC[A/G]CCC |
| | | TGCCCCCCAGCACCCATGGGGC |
| PCSK9 | rs7517090 | GAGTGTGGCCTGTGCAGAAGGGACC[A/G]AG |
| | | GCTGGTGAGACCAGGAGGGCCTG |

Table 5 shows the results of the genotyping experiments using the circulating, cell free DNA isolated by the methods of the present disclosure (designated V) and the two commercially available kits described above (designated C for the NucleoSpin Kit and Q for the QIAamp kit). The data in Table 5 shows that genotyping experiments using circulating cell free DNA purified by the methods of the present disclosure allowed the accurate determination of SNP genotype for all 25 SNPs tested.

TABLE 5

| | Kit C | Kit Q | V |
|---|---|---|---|
| PCSK9, n = 6 | 95.2% | 90.5% | 100% |
| Warfarin Sensitivity, n = 6 | 66.7% | 100% | 100% |
| Plavix Sensitivity, n = 6 | 83.3% | 100% | 100% |
| Thrombophilia Risk, n = 6 | 83.3% | 100% | 100% |

### Example 6—Detection of KRAS Gene Mutations from Circulating, Cell Free DNA

This example illustrates the detection of KRAS gene mutations on circulating cell free DNA isolated using the methods of the present disclosure as well as comparing such detection using circulating cell free DNA isolated using the commercially available kits of Example 4. For the methods of the present disclosure, circulating cell free DNA was prepared as described in for FIG. 1B, with the addition of a desalting step and disassociation of nucleoprotein complexes as described in Example 1. The commercially available kits were used according to manufacturers' instructions. Isolated DNA was used directly for genotyping experiments.

The KRAS mutation is found in several cancers including colorectal, lung, thyroid, and pancreatic cancers and cholangiocarcinoma. KRAS mutations are often located within codons 12 and 13 of exon 2, which may lead to abnormal growth signaling by the p21-ras protein. These alterations in cell growth and division may trigger cancer development as signaling is excessive. A KRAS mutation often serves as a useful prognostic marker of drug response. For example, a KRAS mutation is considered to be a strong prognostic marker of response to tyrosine kinase inhibitors such as gefitinib (Iressa) or erlotinib (Tarceva). Recently, KRAS mutations have been detected in many colorectal cancer patients and may be associated with responses to cetuximab (Erbitux) or panitumumab (Vectibix), which are used in colon cancer therapy Mutations in KRAS codons 12 and 13 have been associated with lack of response to EGFR-targeted therapies in both colorectal cancer and non-small cell lung cancer patients. The National Comprehensive Cancer Network recommends KRAS mutation testing before initiating EGFR-targeted therapies for such diseases.

KRAS mutation analysis was performed using (PNAClamp KRAS Mutation Detection Kit; Panagene, Daejeon, Korea). The mutation analysis was performed according to manufactures' instructions. Table 6 shows the results of the KRAS mutation experiments using the circulating, cell free DNA isolated by the methods of the present disclosure (designated V) and the two commercially available kits described above (designated C for the NucleoSpin Kit and Q for the QIAamp kit). PCR conditions were: 94° C.^{5 min}×1 cycle; (94° C.^{30sec}−70° C.^{20sec}−63° C.^{30sec}−72° C.^{30sec})×40 cycles.

The data in Table 6 shows that KRAS mutation analysis using circulating cell free DNA purified by the methods of the present disclosure allowed the accurate determination of KRAS mutation status that was superior to the results obtained with commercially available kit Q.

TABLE 6

| | Kit C | Kit Q | V |
|---|---|---|---|
| KRAS Mutation | ND | 4/14 detected | 9/14 detected |
| Status (Codon | | | |
| 12/13); n = 7 | | | |

### Example 7—Detection of TP53 Exon Amplification Products from Circulating, Cell Free DNA

This example illustrates the detection of TP53 exon amplification products from circulating cell free DNA isolated using the methods of the present disclosure as well as comparing such detection using circulating cell free DNA isolated using the commercially available kits of Example 4. For the methods of the present disclosure, circulating cell free DNA was prepared as described in for FIG. 1B, with the addition of a desalting step and disassociation of nucleoprotein complexes as described in Example 1. The commercially available kits were used according to manufacturers' instructions. Isolated DNA was used directly for genotyping experiments. The TP53 gene provides instructions for making tumor protein p53. This protein acts as a tumor suppressor, which regulates cell division by keeping cells from growing and dividing too fast or in an uncontrolled way. Identifying mutations in the TP53 gene is important for diagnostic, prognostic and monitoring purposes and can guide in the selection of appropriate therapeutic intervention. TP53 mutations are found associated with drug resistance to platinum-based chemotherapy without any effect on the sensitivity of paclitaxel. Thus, detection and analysis of gene mutations of TP53 have become a guide for cancer individualized chemotherapy.

TP53 exon amplification analysis was performed on exon 1 of TP53 using (Multiplex PCR kit for Human TP53 Oncogene from Bio SB). The analysis was performed according to manufactures' instructions. PCR conditions were: 95° C.^{5 min}×1 cycle; (95° C.^{25sec}−64° C.^{20sec}−72° C.^{20sec})×15 cycles; (93° C.^{25sec}−60° C.^{35sec}−72° C.^{20sec})×28 cycles.

Table 7 shows the results of the TP53 exon amplification experiments using the circulating, cell free DNA isolated by the methods of the present disclosure (designated V) and the two commercially available kits described above (designated C for the NucleoSpin Kit and Q for the QIAamp kit). The data in Table 7 shows that exon amplification of the TP53 gene using circulating cell free DNA purified by the methods of the present disclosure was equal to (Kit Q) or superior to (Kit C) the results obtained with commercially available kit Q.

TABLE 7

| | Kit C | Kit Q | V |
|---|---|---|---|
| TP53 Exon | + | +++ | +++ |
| Amplification (392- | | | |
| 1,143 bp), n = 6 | | | |

### Example 8—Detection of Short Tandem Repeat DNA Profiling from Circulating, Cell Free DNA

This example illustrates the detection of short tandem repeat (STR) profiling from circulating cell free DNA isolated using the methods of the present disclosure as well as comparing such detection using circulating cell free DNA isolated using the commercially available kits of Example 4. STRs are highly polymorphic, repeating sequences and represent non-coding DNA sequences. STRs are frequently used in determining identity. As STRs are non-coding DNA regions, the nucleic acid containing such STRs should not be enriched in the methods of the present disclosure. For the methods of the present disclosure, circulating cell free DNA was prepared as described in for FIG. 1B, with the addition of a desalting step and disassociation of nucleoprotein complexes as described in Example 1. The commercially available kits were used according to manufacturers' instructions. Isolated DNA was used directly for genotyping experiments.

16 STRs were analyzed using the AmpFLSTR Identifiler PCR Amplification kit. This kit allows the determination of 15 tetranucleotide repeat loci (D8S1179, D21S11, D7S820, CSF1PO, D351358, TH01, D13S317, D165539, D21S1338, D195433, vWA, TPDX, D18S51, D5S818, FGA and the Amelogenin gender-determining marker) in a single PCR amplification. Reactions were carried out according to manufacturers' instructions on circulating, cell free DNA isolated using the methods of the present disclosure and circulating, cell free DNA isolated using the commercial kits described above. PCR conditions were: 95° C.^{11min}×1 cycle; (94° C.^{60sec}−59° C.^{60sec}−72° C.^{60sec})×28 cycles; 60° C.^{60min}×1 cycle.

Table 8 shows the results of the STR profiling experiments using the circulating, cell free DNA isolated by the methods of the present disclosure (designated V) and the two commercially available kits described above (designated C for the NucleoSpin Kit and Q for the QIAamp kit). The data in Table 8 shows that STR profiling using circulating cell free DNA isolated using the methods of the present disclosure was less effective, as expected, than using circulating cell free DNA isolated using Kit Q in detection of these non-coding STR markers.

TABLE 8
(;

| | Kit C | Kit Q | V |
|---|---|---|---|
| STR DNA Profiling; | ND | 6/16, 9/15, | 0/16, 8/15, |
| n = 4 | | 4/16, 0/16 | 1/16, 0/16 |

### Example 9—Array Comparative Genomic Hybridization from Circulating, Cell Free DNA

This example illustrates the use of array comparative genomic hybridization (aCGH) from circulating cell free DNA isolated using the methods of the present disclosure as well as comparing such detection using circulating cell free DNA isolated using the commercially available kits of Example 4. For the methods of the present disclosure, circulating cell free DNA was prepared as described in for FIG. 1B, with the addition of a desalting step and disassociation of nucleoprotein complexes as described in Example 1. The commercially available kits were used according to manufacturers' instructions. Isolated DNA was used directly for genotyping experiments.

In this example, the InfiniumDx CytoSNP-12 Assay (Illumina) was used to screen over 294,000 SNP markers over the entire genome. Reactions were carried out according to manufacturers' instructions on circulating, cell free DNA isolated using the methods of the present disclosure and circulating, cell free DNA isolated using the commercial kits described above. aCGH analysis is useful in detecting genomic structural variation such as copy number changes, copy neutral loss of heterozygosity, uniparental disomy, mitotic recombination, gene conversion and mosaicism.

Table 9 shows the results of the aCGH experiments using the circulating, cell free DNA isolated by the methods of the present disclosure (designated V) and the two commercially available kits described above (designated C for the NucleoSpin Kit and Q for the QIAamp kit). The data in Table 9 shows that aCGH SNP profiling of whole human genome using circulating cell free DNA purified by the methods of the present disclosure was more representative of cell genomic DNA than using circulating cell free DNA isolated with Kit Q.

TABLE 9

| | Kit C | Kit Q | V |
|---|---|---|---|
| Array GCH | ND | Poorly | Highly |
| Analysis; n = 4 | | Representative | Representative |

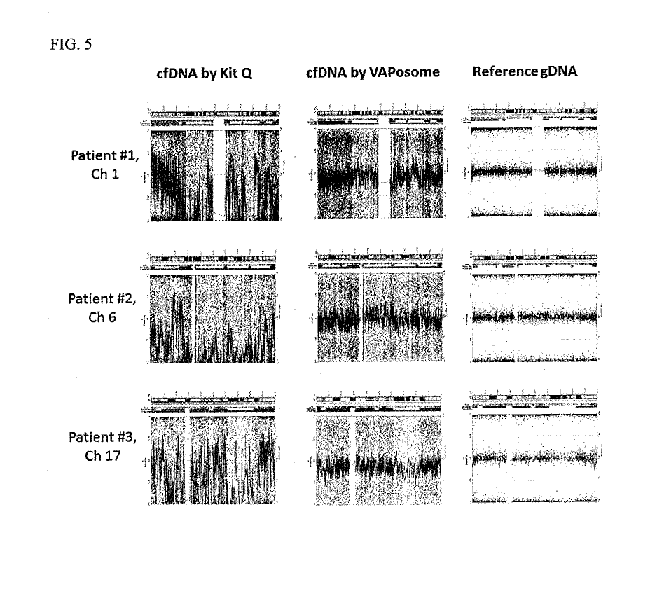

FIG. 5 shows representative results of aCGH of individual chromosomes using circulating cell free DNA isolated using the methods of the present disclosure as well as comparing such detection using circulating cell free DNA isolated using the commercially available Kit Q of Example 4. The individual chromosomes shown are chromosome 1 (upper panel), chromosome 6 (middle panel) and chromosome 17 (lower panel). The leftmost column shows the aCGH analysis performed using circulating, cell free DNA isolated using Kit Q, the middle column shows the aCGH analysis performed using circulating, cell free DNA isolated using the methods of the present disclosure and the rightmost column shows the aCGH analysis performed using genomic DNA (reference gDNA). As can be seen, the results obtained using circulating, cell free DNA isolated by the method of the present disclosure provided superior results when compared to results obtained using circulating, cell free DNA isolated by Kit Q and were in agreement with the results obtained using genomic DNA.

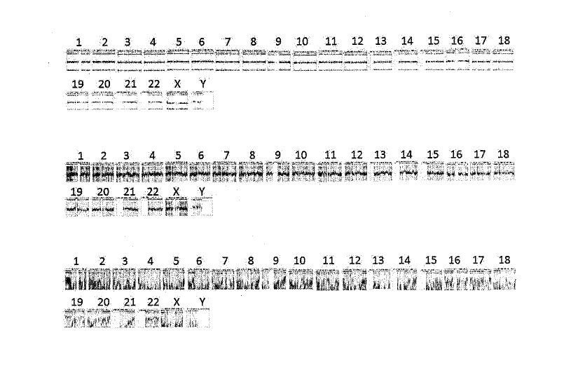

FIG. 6 shows the results from all 22 autosomes and each allosome. The lower panel shows the aCGH analysis performed using circulating, cell free DNA isolated using Kit Q, the middle panel shows the aCGH analysis performed using circulating, cell free DNA isolated using the methods of the present disclosure and the upper panel shows the aCGH analysis performed using genomic DNA (reference gDNA). As can be seen, the results obtained using circulating, cell free DNA isolated by the method of the present disclosure provided superior results when compared to results obtained using circulating, cell free DNA isolated by Kit Q and were in agreement with the results obtained using genomic DNA.

The results in Table 9 and FIGS. 5 and 6 show that the nucleic acid isolated using the methods of the present disclosure is highly representative of genomic DNA.

### Example 10—Analysis of Lipid and Polypeptide Composition

As discussed herein, the nucleic acids isolated according to the methods of the present disclosure are associated with cellular components, such as but not limited to, polypeptides and lipids. These associated cellular components aid in protecting the nucleic acids from degradation and provide an additional source of information regarding the subject. As a result, the biological information that can be obtained from the material isolated as described herein is greater than obtainable with commercially available kits which extract only the nucleic acids. To investigate the nature of the associated cellular components that are isolated with the circulating cell free nucleic acid, circulating cell free nucleic acid and its associated cellular components were isolated as described for FIG. 1B.

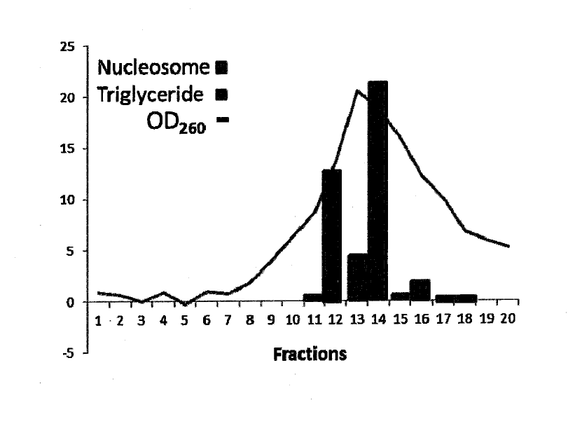

The isolated material was subject to Vertical Auto Profile (VAP) analysis as described in Example 1 to identify the nature of the lipid component associated with the nucleic acid. Nucleic acid/histone complexes were detected in density gradient fractions using the Cell Death Detection ELISA kit (Roche, catalogue number 1774425). This assay detects histone associated DNA complexes, both mono- and loig-onucleosomes, using a photometric enzyme immunoassay and was used to determine the fractions containing DNA associated with cellular components. As shown in FIG. 7, triglyceride was the only lipid detected in the isolated complexes, with the triglyceride being detected only in those fractions where polypeptide associated DNA was detected. The solid line in FIG. 7 indicates absorbance at 260 nm.

In addition, polypeptide components associated with the isolated nucleic acid were analyzed by LC-MS/MS analysis to determine the identity of the associated polypeptides. As shown in Table 10, over 700 associated polypeptides were identified, indicating that a variety of polypeptides are associated with the isolated nucleic acids.

TABLE 10

| | | | Accession | Molecular |
|---|---|---|---|---|
| Identified Proteins | Taxonomy | RepID | Number | Weight |
| 14-3-3 protein epsilon | Amniota | 1433E_HUMAN | P62258 | 29 kDa |
| 14-3-3 protein eta | Simiiformes | 1433F_HUMAN | Q04917 | 28 kDa |
| | | | (+1) | |
| 14-3-3 protein gamma | Amniota | 1433G_HUMAN | P61981 | 28 kDa |
| 14-3-3 protein sigma | Hominoidea | 1433S_HUMAN | P31947 | 28 kDa |
| 14-3-3 protein theta | Eutheria | 1433T_HUMAN | P27348 | 28 kDa |
| 14-3-3 protein zeta/delta | Amniota | 1433Z_HUMAN | P63104 | 28 kDa |
| 26S protease regulatory | Homo sapiens | E9PM69_HUMAN | E9PM69 | 44 kDa |
| subunit 6A | | | (+1) | |
| 26S protease regulatory | Eutheria | PRS7_HUMAN | P35998 | 49 kDa |
| subunit 7 | | | | |
| 26S protease regulatory | Eutheria | A8K3Z3_HUMAN | A8K3Z3 | 45 kDa |
| subunit 8 | | | (+1) | |
| 26S proteasome non-ATPase | Homo sapiens | PSMD1_HUMAN | Q99460 | 106 kDa |
| regulatory subunit 1 | | | | |
| 26S proteasome non-ATPase | Catarrhini | PSD12_HUMAN | O00232 | 53 kDa |
| regulatory subunit 12 | | | | |
| 26S proteasome non-ATPase | Homo sapiens | B4DJ66_HUMAN | B4DJ66 | 35 kDa |
| regulatory subunit 13 | | | (+2) | |
| 26S proteasome non-ATPase | Homininae | PSMD2_HUMAN | Q13200 | 100 kDa |
| regulatory subunit 2 | | | (+4) | |
| 26S proteasome non-ATPase | Homininae | C9IZE4_HUMAN | C9IZE4 | 52 kDa |
| regulatory subunit 6 | | | (+1) | |
| 28S ribosomal protein S9, | Homo sapiens | RT09_HUMAN | P82933 | 46 kDa |
| mitochondrial | | | | |
| 3-hydroxyacyl-CoA | Homininae | HCD2_HUMAN | Q99714 | 27 kDa |
| dehydrogenase type-2 | | | | |
| 3-ketoacyl-CoA thiolase | Catarrhini | B4E2W0_HUMAN | B4E2W0 | 49 kDa |
| | | | (+1) | |
| 40S ribosomal protein S11 | Amniota | RS11_HUMAN | P62280 | 18 kDa |
| 40S ribosomal protein S12 | Mammalia | RS12_HUMAN | P25398 | 15 kDa |
| 40S ribosomal protein S14 | Euteleostomi | RS14_HUMAN | P62263 | 16 kDa |
| 40S ribosomal protein S15a | Mammalia | RS15A_HUMAN | P62244 | 15 kDa |
| 40S ribosomal protein S18 | Euarchontoglires | Q5GGW2_HUMAN | Q5GGW2 | 4 kDa |
| 40S ribosomal protein S2 | Homo sapiens | E9PQD7_HUMAN | E9PQD7 | 25 kDa |
| | | | (+2) | |
| 40S ribosomal protein S20 | Homo sapiens | E5RIP1_HUMAN | E5RIP1 | 5 kDa |
| | | | (+2) | |
| 40S ribosomal protein S24 | Homo sapiens | E7EPK6_HUMAN | E7EPK6 | 32 kDa |
| | | | (+1) | |
| 40S ribosomal protein S25 | Amniota | RS25_HUMAN | P62851 | 14 kDa |
| 40S ribosomal protein S3 | Eukaryota | RS3_HUMAN | P23396 | 27 kDa |
| 40S ribosomal protein S3a | Eutheria | RS3A_HUMAN | P61247 | 30 kDa |
| 40S ribosomal protein S4, X | Eutheria | RS4X_HUMAN | P62701 | 30 kDa |
| isoform | | | | |
| 40S ribosomal protein S5 | Mammalia | RS5_HUMAN | P46782 | 23 kDa |
| 40S ribosomal protein S7 | Theria | RS7_HUMAN | P62081 | 22 kDa |
| 40S ribosomal protein S9 | Eutheria | RS9_HUMAN | P46781 | 23 kDa |
| 4F2 cell-surface antigen | Homo sapiens | B4E2Z3_HUMAN | B4E2Z3 | 56 kDa |
| heavy chain | | | (+2) | |
| 5-aminoimidazole-4- | Homo sapiens | A8K202_HUMAN | A8K202 | 65 kDa |
| carboxamide ribonucleotide | | | (+1) | |
| formyltransferase/IMP | | | | |
| cyclohydrolase, isoform | | | | |
| CRA_g | | | | |
| 5-azacytidine induced 1 | Homo sapiens | B2RN10_HUMAN | B2RN10 | 122 kDa |
| | | | (+1) | |
| 60 kDa heat shock protein, | Homo sapiens | CH60_HUMAN | P10809 | 61 kDa |
| mitochondrial | | | | |
| 60S acidic ribosomal protein | Homo sapiens | F8VWS0_HUMAN | F8VWS0 | 30 kDa |
| P0 | | | (+2) | |
| 60S acidic ribosomal protein | Catarrhini | RLA2_HUMAN | P05387 | 12 kDa |
| P2 | | | | |
| 60S ribosomal protein L10 | Euarchontoglires | F8W7C6_HUMAN | F8W7C6 | 19 kDa |
| | | | (+1) | |
| 60S ribosomal protein L10a | Eutheria | RL10A_HUMAN | P62906 | 25 kDa |
| 60S ribosomal protein L12 | Eutheria | RL12_HUMAN | P30050 | 18 kDa |
| 60S ribosomal protein L13 | Homo sapiens | RL13_HUMAN | P26373 | 24 kDa |
| 60S ribosomal protein L13a | Hominidae | RL13A_HUMAN | P40429 | 24 kDa |
| | | | (+1) | |
| 60S ribosomal protein L17 | Homininae | B4E3C2_HUMAN | B4E3C2 | 17 kDa |
| | | | (+1) | |
| 60S ribosomal protein L18 | Homo sapiens | B4DDY5_HUMAN | B4DDY5 | 16 kDa |
| | | | (+3) | |
| 60S ribosomal protein L18a | Homo sapiens | B2R4C0_HUMAN | B2R4C0 | 21 kDa |
| | | | (+2) | |
| 60S ribosomal protein L19 | Theria | RL19_HUMAN | P84098 | 23 kDa |
| 60S ribosomal protein L23 | Euteleostomi | RL23_HUMAN | P62829 | 15 kDa |
| 60S ribosomal protein L23a | Eutheria | RL23A_HUMAN | P62750 | 18 kDa |
| 60S ribosomal protein L24 | Eutheria | RL24_HUMAN | P83731 | 18 kDa |
| 60S ribosomal protein L28 | Catarrhini | RL28_HUMAN | P46779 | 16 kDa |
| 60S ribosomal protein L3 | Catarrhini | RL3_HUMAN | P39023 | 46 kDa |
| 60S ribosomal protein L32 | Homininae | F8W727_HUMAN | F8W727 | 18 kDa |
| | | | (+1) | |
| 60S ribosomal protein L4 | Homo sapiens | E7EWF1_HUMAN | E7EWF1 | 46 kDa |
| | | | (+1) | |
| 60S ribosomal protein L5 | Hominoidea | RL5_HUMAN | P46777 | 34 kDa |
| 60S ribosomal protein L6 | Homininae | RL6_HUMAN | Q02878 | 33 kDa |
| 60S ribosomal protein L7 | Homininae | RL7_HUMAN | P18124 | 29 kDa |
| 60S ribosomal protein L7a | Eutheria | RL7A_HUMAN | P62424 | 30 kDa |
| 60S ribosomal protein L8 | Homo sapiens | E9PIZ3_HUMAN | E9PIZ3 | 24 kDa |
| | | | (+1) | |
| 6-phosphofructokinase | Homo sapiens | Q6ZTT1_HUMAN | Q6ZTT1 | 93 kDa |
| | | | (+1) | |
| 6-phosphofructokinase type | Homo sapiens | K6PP_HUMAN | Q01813 | 86 kDa |
| C | | | | |
| 6-phosphofructokinase, liver | Homo sapiens | K6PL_HUMAN | P17858 | 85 kDa |
| type | | | | |
| 6-phosphogluconate | Homo sapiens | 6PGD_HUMAN | P52209 | 53 kDa |
| dehydrogenase, | | | (+1) | |
| decarboxylating | | | | |
| 78 kDa glucose-regulated | Catarrhini | GRP78_HUMAN | P11021 | 72 kDa |
| protein | | | | |
| 7-dehydrocholesterol | Homo sapiens | DHCR7_HUMAN | Q9UBM7 | 54 kDa |
| reductase | | | | |
| ABC50 protein | Homo sapiens | Q2L6I2_HUMAN | Q2L6I2 | 92 kDa |
| | | | (+1) | |
| Abhydrolase domain- | Homo sapiens | B4DNR3_HUMAN | B4DNR3 | 20 kDa |
| containing protein 14B | | | (+2) | |
| Acetolactate synthase-like | Homo sapiens | ILVBL_HUMAN | A1L0T0 | 68 kDa |
| protein | | | | |
| Acetyl-CoA | Homininae | THIL_HUMAN | P24752 | 45 kDa |
| acetyltransferase, | | | | |
| mitochondrial | | | | |
| Aconitase 2, mitochondrial | Homo sapiens | A2A274_HUMAN | A2A274 | 88 kDa |
| | | | (+2) | |
| Actin, cytoplasmic 1, N- | Simiiformes | B4E335_HUMAN | B4E335 | 39 kDa |
| terminally processed | | | (+3) | |
| Actin-related protein 2/3 | Homo sapiens | C9JWM7_HUMAN | C9JWM7 | 22 kDa |
| complex subunit 4 | | | (+3) | |
| ADAM metallopeptidase | Catarrhini | A8MY20_HUMAN | A8MY20 | 63 kDa |
| domain 10, isoform CRA_a | | | (+1) | |
| Adaptor-related protein | Homo sapiens | H0UID3_HUMAN | H0UID3 | 105 kDa |
| complex 2, beta 1 subunit, | | | (+3) | |
| isoform CRA_d | | | | |
| Adenine | Homo sapiens | H3BQZ9_HUMAN | H3BQZ9 | 17 kDa |
| phosphoribosyltransferase | | | (+1) | |
| Adenosylhomocysteinase | Homininae | SAHH_HUMAN | P23526 | 48 kDa |
| | | | (+1) | |
| Adenylosuccinate synthetase | Homo sapiens | B4E1L0_HUMAN | B4E1L0 | 48 kDa |
| | | | (+1) | |
| Adenylyl cyclase-associated | Homo sapiens | B2RDY9_HUMAN | B2RDY9 | 52 kDa |
| protein | | | (+1) | |
| ADP/ATP translocase 2 | Homo sapiens | ADT2_HUMAN | P05141 | 33 kDa |
| | | | (+1) | |
| ADP-ribosylation factor 3 | Euteleostomi | ARF3_HUMAN | P61204 | 21 kDa |
| | | | (+2) | |
| ADP-ribosylation factor 6 | Homo sapiens | Q5U025_HUMAN | Q5U025 | 20 kDa |
| Afamin | Homo sapiens | AFAM_HUMAN | P43652 | 69 kDa |
| A-kinase anchor protein 13 | Homo sapiens | H0Y4V5_HUMAN | H0Y4V5 | 305 kDa |
| | | | (+3) | |
| Alanine--tRNA ligase, | Homo sapiens | SYAC_HUMAN | P49588 | 107 kDa |
| cytoplasmic | | | | |
| Alcohol dehydrogenase class- | Homo sapiens | ADHX_HUMAN | P11766 | 40 kDa |
| 3 | | | (+2) | |
| Aldehyde dehydrogenase X, | Homo sapiens | AL1B1_HUMAN | P30837 | 57 kDa |
| mitochondrial | | | | |
| Aldose reductase | Homo sapiens | ALDR_HUMAN | P15121 | 36 kDa |
| Alpha-1-antichymotrypsin | Homo sapiens | AACT_HUMAN | P01011 | 48 kDa |
| Alpha-1-antitrypsin | Homo sapiens | A1AT_HUMAN | P01009 | 47 kDa |
| Alpha-1B-glycoprotein | Homo sapiens | A1BG_HUMAN | P04217 | 54 kDa |
| Alpha-2-antiplasmin | Homininae | A2AP_HUMAN | P08697 | 55 kDa |
| Alpha-2-macroglobulin | Homo sapiens | A2MG_HUMAN | P01023 | 163 kDa |
| Alpha-actinin-1 | Homo sapiens | ACTN1_HUMAN | P12814 | 103 kDa |
| Alpha-actinin-4 | Catarrhini | ACTN4_HUMAN | O43707 | 105 kDa |
| Alpha-aminoadipic | Homo sapiens | E7EPT3_HUMAN | E7EPT3 | 54 kDa |
| semialdehyde dehydrogenase | | | (+1) | |
| Alpha-enolase | Catarrhini | ENOA_HUMAN | P06733 | 47 kDa |
| Aminoacyl tRNA synthase | Homo sapiens | AIMP1_HUMAN | Q12904 | 34 kDa |
| complex-interacting | | | | |
| multifunctional protein 1 | | | | |
| Aminoacyl tRNA synthase | Homo sapiens | F8W950_HUMAN | F8W950 | 28 kDa |
| complex-interacting | | | (+2) | |
| multifunctional protein 2 | | | | |
| Aminopeptidase B | Homo sapiens | AMPB_HUMAN | Q9H4A4 | 73 kDa |
| Angiotensinogen | Homo sapiens | ANGT_HUMAN | P01019 | 53 kDa |
| | | | (+2) | |
| Ankyrin repeat domain- | Homo sapiens | ANR31_HUMAN | Q8N7Z5 | 211 kDa |
| containing protein 31 | | | | |
| Ankyrin-3 | Homo sapiens | ANK3_HUMAN | Q12955 | 480 kDa |
| Annexin | Homo sapiens | B7Z8A7_HUMAN | B7Z8A7 | 72 kDa |
| | | | (+1) | |
| Annexin | Homo sapiens | B2R657_HUMAN | B2R657 | 53 kDa |
| Annexin A1 | Homininae | ANXA1_HUMAN | P04083 | 39 kDa |
| Annexin A2 | Catarrhini | ANXA2_HUMAN | P07355 | 39 kDa |
| Annexin A3 | Homo sapiens | ANXA3_HUMAN | P12429 | 36 kDa |
| Annexin A4 | Homo sapiens | ANXA4_HUMAN | P09525 | 36 kDa |
| Annexin A5 | Homininae | ANXA5_HUMAN | P08758 | 36 kDa |
| Antithrombin-III | Homo sapiens | ANT3_HUMAN | P01008 | 53 kDa |
| AP-3 complex subunit beta-1 | Homo sapiens | AP3B1_HUMAN | O00203 | 121 kDa |
| AP-3 complex subunit mu-1 | Eutheria | AP3M1_HUMAN | Q9Y2T2 | 47 kDa |
| Apolipoprotein A-I | Homininae | APOA1_HUMAN | P02647 | 31 kDa |
| Apolipoprotein A-II | Homininae | APOA2_HUMAN | P02652 | 11 kDa |
| Apolipoprotein A-IV | Homo sapiens | APOA4_HUMAN | P06727 | 45 kDa |
| Apolipoprotein B (Including | Homo sapiens | C0JYY2_HUMAN | C0JYY2 | 516 kDa |
| Ag(X) antigen) | | | | |
| Apolipoprotein C-III | Homo sapiens | APOC3_HUMAN | P02656 | 11 kDa |
| Apolipoprotein D | Homo sapiens | APOD_HUMAN | P05090 | 21 kDa |
| Apolipoprotein E | Homo sapiens | APOE_HUMAN | P02649 | 36 kDa |
| Apolipoprotein F | Homo sapiens | APOF_HUMAN | Q13790 | 35 kDa |
| Apolipoprotein L1 | Homo sapiens | E9PF24_HUMAN | E9PF24 | 42 kDa |
| | | | (+1) | |
| Apolipoprotein M | Homo sapiens | APOM_HUMAN | O95445 | 21 kDa |
| | | | (+2) | |
| Apolipoprotein(a) | Catarrhini | APOA_HUMAN | P08519 | 501 kDa |
| | | | (+1) | |
| Arginine--tRNA ligase, | Homo sapiens | SYRC_HUMAN | P54136 | 75 kDa |
| cytoplasmic | | | | |
| Asparagine synthetase | Homo sapiens | ASNS_HUMAN | P08243 | 64 kDa |
| [glutamine-hydrolyzing] | | | (+1) | |
| Asparagine--tRNA ligase, | Homo sapiens | SYNC_HUMAN | O43776 | 63 kDa |
| cytoplasmic | | | | |
| Aspartate aminotransferase, | Homo sapiens | AATM_HUMAN | P00505 | 48 kDa |
| mitochondrial | | | (+1) | |
| Aspartate--tRNA ligase, | Homo sapiens | SYDC_HUMAN | P14868 | 57 kDa |
| cytoplasmic | | | | |
| Aspartyl/asparaginyl beta- | Homo sapiens | ASPH_HUMAN | Q12797 | 86 kDa |
| hydroxylase | | | | |
| Atlastin-3 | Homininae | F5H6I7_HUMAN | F5H6I7 | 59 kDa |
| | | | (+1) | |
| ATP synthase subunit alpha | Homo sapiens | B4DY56_HUMAN | B4DY56 | 58 kDa |
| | | | (+1) | |
| ATP synthase subunit beta, | Eutheria | ATPB_HUMAN | P06576 | 57 kDa |
| mitochondrial | | | | |
| ATP-binding cassette sub- | Homininae | E7EUE1_HUMAN | E7EUE1 | 78 kDa |
| family D member 3 | | | (+1) | |
| ATP-binding cassette sub- | Euarchontoglires | ABCE1_HUMAN | P61221 | 67 kDa |
| family E member 1 | | | | |
| ATP-citrate synthase | Homo sapiens | ACLY_HUMAN | P53396 | 121 kDa |
| ATP-dependent RNA | Homininae | DHX9_HUMAN | Q08211 | 141 kDa |
| helicase A | | | | |
| ATP-dependent RNA | Homo sapiens | DDX25_HUMAN | Q9UHL0 | 55 kDa |
| helicase DDX25 | | | (+1) | |
| ATP-dependent RNA | Homo sapiens | B5BTY4_HUMAN | B5BTY4 | 73 kDa |
| helicase DDX3X | | | (+1) | |
| ATP-dependent RNA | Homo sapiens | DHX29_HUMAN | Q7Z478 | 155 kDa |
| helicase DHX29 | | | | |
| AT-rich interactive domain- | Homo sapiens | ARI4A_HUMAN | P29374 | 143 kDa |
| containing protein 4A | | | | |
| Basic leucine zipper and W2 | Eutheria | BZW1_HUMAN | Q7L1Q6 | 48 kDa |
| domain-containing protein 1 | | | | |
| Basigin | Homo sapiens | BASI_HUMAN | P35613 | 42 kDa |
| | | | (+1) | |
| Beta-2-glycoprotein 1 | Homo sapiens | APOH_HUMAN | P02749 | 38 kDa |
| Beta-lactamase-like protein 2 | Homo sapiens | LACB2_HUMAN | Q53H82 | 33 kDa |
| Bifunctional | Homo sapiens | SYEP_HUMAN | P07814 | 171 kDa |
| glutamate/proline--tRNA | | | | |
| ligase | | | | |
| Brain acid soluble protein 1 | Homo sapiens | BASP1_HUMAN | P80723 | 23 kDa |
| BRCA1-A complex subunit | Simiiformes | BRE_HUMAN | Q9NXR7 | 44 kDa |
| BRE | | | | |
| Brefeldin A-inhibited | Homo sapiens | BIG1_HUMAN | Q9Y6D6 | 209 kDa |
| guanine nucleotide-exchange | | | | |
| protein 1 | | | | |
| C-1-tetrahydrofolate | Homo sapiens | C1TC_HUMAN | P11586 | 102 kDa |
| synthase, cytoplasmic | | | | |
| C4b-binding protein alpha | Homo sapiens | C4BPA_HUMAN | P04003 | 67 kDa |
| chain | | | | |
| CAD protein | Homo sapiens | PYR1_HUMAN | P27708 | 243 kDa |
| Calnexin | Homo sapiens | CALX_HUMAN | P27824 | 68 kDa |
| Calpain small subunit 1 | Hominidae | CPNS1_HUMAN | P04632 | 28 kDa |
| Calpain-1 catalytic subunit | Homo sapiens | CAN1_HUMAN | P07384 | 82 kDa |
| Calpain-2 catalytic subunit | Homo sapiens | H0Y323_HUMAN | H0Y323 | 83 kDa |
| | | | (+1) | |
| Calponin-2 | Homo sapiens | B4DDF4_HUMAN | B4DDF4 | 33 kDa |
| | | | (+3) | |
| Calponin-3 | Homo sapiens | F8WA86_HUMAN | F8WA86 | 31 kDa |
| | | | (+1) | |
| cAMP-dependent protein | Hominoidea | KAP0_HUMAN | P10644 | 43 kDa |
| kinase type I-alpha regulatory | | | | |
| subunit | | | | |
| Caprin-1 | Catarrhini | CAPR1_HUMAN | Q14444 | 78 kDa |
| Carbonyl reductase | Homininae | CBR1_HUMAN | P16152 | 30 kDa |
| [NADPH] 1 | | | | |
| Carboxypeptidase N subunit 2 | Homo sapiens | CPN2_HUMAN | P22792 | 61 kDa |
| Casein kinase II subunit | Eutheria | CSK21_HUMAN | P68400 | 45 kDa |
| alpha | | | (+3) | |
| Caspase recruitment domain- | Homo sapiens | CAR11_HUMAN | Q9BXL7 | 133 kDa |
| containing protein 11 | | | | |
| Catenin alpha-1 | Homo sapiens | F6XBD8_HUMAN | F6XBD8 | 103 kDa |
| | | | (+1) | |
| Catenin beta-1 | Homo sapiens | B5BU28_HUMAN | B5BU28 | 86 kDa |
| | | | (+1) | |
| Cathepsin D | Homo sapiens | CATD_HUMAN | P07339 | 45 kDa |
| CCT7 protein | Homo sapiens | Q6IBT3_HUMAN | Q6IBT3 | 59 kDa |
| | | | (+1) | |
| cDNA PSEC0119 fis, clone | Homo sapiens | Q8NBL9_HUMAN | Q8NBL9 | 62 kDa |
| PLACE1002376, highly | | | (+1) | |
| similar to GPI transamidase | | | | |
| component PIG-S | | | | |
| Cell division control protein | Theria | CDC42_HUMAN | P60953 | 21 kDa |
| 42 homolog | | | | |
| Cell migration-inducing | Homo sapiens | A1KYQ7_HUMAN | A1KYQ7 | 105 kDa |
| protein 17 | | | (+1) | |
| Cellular apoptosis | Homo sapiens | A3RLL6_HUMAN | A3RLL6 | 104 kDa |
| susceptibility protein variant | | | (+1) | |
| 2 | | | | |
| Centromere protein F | Homo sapiens | CENPF_HUMAN | P49454 | 368 kDa |
| Ceruloplasmin | Homo sapiens | CERU_HUMAN | P00450 | 122 kDa |
| Chaperonin containing TCP1, | Homo sapiens | G5E9B2_HUMAN | G5E9B2 | 59 kDa |
| subunit 8 (Theta), isoform | | | (+1) | |
| CRA_a | | | | |
| Chloride intracellular channel | Homininae | CLIC1_HUMAN | O00299 | 27 kDa |
| protein 1 | | | | |
| Chromobox protein homolog | Eutheria | CBX3_HUMAN | Q13185 | 21 kDa |
| 3 | | | | |
| Citrate synthase, | Homo sapiens | CISY_HUMAN | O75390 | 52 kDa |
| mitochondrial | | | (+1) | |
| Citron | Homo sapiens | Q2M5E1_HUMAN | Q2M5E1 | 237 kDa |
| C-Jun-amino-terminal | Catarrhini | JIP4_HUMAN | O60271 | 146 kDa |
| kinase-interacting protein 4 | | | | |
| Clathrin heavy chain 1 | Eutheria | CLH1_HUMAN | Q00610 | 192 kDa |
| Clusterin | Homo sapiens | CLUS_HUMAN | P10909 | 52 kDa |
| Coactosin-like protein | Hominidae | COTL1_HUMAN | Q14019 | 16 kDa |
| Coagulation factor XIII A | Homo sapiens | F13A_HUMAN | P00488 | 83 kDa |
| chain | | | (+1) | |
| Coatomer subunit alpha | Homo sapiens | COPA_HUMAN | P53621 | 138 kDa |
| Coatomer subunit beta | Homo sapiens | COPB_HUMAN | P53618 | 107 kDa |
| Coatomer subunit beta′ | Homo sapiens | COPB2_HUMAN | P35606 | 102 kDa |
| Coatomer subunit gamma | Homo sapiens | B3KND4_HUMAN | B3KND4 | 76 kDa |
| | | | (+2) | |
| Coatomer subunit gamma-1 | Homininae | COPG1_HUMAN | Q9Y678 | 98 kDa |
| Cofilin 1 (Non-muscle), | Homo sapiens | G3V1A4_HUMAN | G3V1A4 | 17 kDa |
| isoform CRA_a | | | (+1) | |
| COL21A1 protein | Homo sapiens | B7ZLK3_HUMAN | B7ZLK3 | 99 kDa |
| | | | (+2) | |
| Complement C1q | Homo sapiens | C1QB_HUMAN | P02746 | 27 kDa |
| subcomponent subunit B | | | | |
| Complement C1q | Homo sapiens | C1QC_HUMAN | P02747 | 26 kDa |
| subcomponent subunit C | | | | |
| Complement C1s | Homo sapiens | A6NG18_HUMAN | A6NG18 | 76 kDa |
| subcomponent heavy chain | | | (+1) | |
| Complement C3 | Homo sapiens | CO3_HUMAN | P01024 | 187 kDa |
| Complement C5 | Homo sapiens | CO5_HUMAN | P01031 | 188 kDa |
| Complement component 4A | Homo sapiens | A7E2V2_HUMAN | A7E2V2 | 193 kDa |
| (Rodgers blood group) | | | (+7) | |
| Complement component C7 | Homo sapiens | CO7_HUMAN | P10643 | 94 kDa |
| Complement component C8 | Homo sapiens | CO8G_HUMAN | P07360 | 22 kDa |
| gamma chain | | | | |
| Complement component C9 | Homo sapiens | CO9_HUMAN | P02748 | 63 kDa |
| Complement factor B | Homo sapiens | B4E1Z4_HUMAN | B4E1Z4 | 141 kDa |
| Complement factor H | Homo sapiens | CFAH_HUMAN | P08603 | 139 kDa |
| Complement factor I | Homo sapiens | CFAI_HUMAN | P05156 | 66 kDa |
| | | | (+2) | |
| Complement factor | Homo sapiens | B3KVK6_HUMAN | B3KVK6 | 39 kDa |
| properdin, isoform CRA_c | | | (+1) | |
| COP9 constitutive | Homo sapiens | B3KST5_HUMAN | B3KST5 | 40 kDa |
| photomorphogenic homolog | | | (+2) | |
| subunit 4 (Arabidopsis), | | | | |
| isoform CRA_a | | | | |
| Copine I | Homo sapiens | A6PVH9_HUMAN | A6PVH9 | 53 kDa |
| | | | (+2) | |
| Creatine kinase B-type | Homo sapiens | E7EUJ8_HUMAN | E7EUJ8 | 39 kDa |
| | | | (+1) | |
| Cullin-4B | Homo sapiens | CUL4B_HUMAN | Q13620 | 104 kDa |
| | | | (+1) | |
| Cullin-associated NEDD8- | Eutheria | CAND1_HUMAN | Q86VP6 | 136 kDa |
| dissociated protein 1 | | | | |
| Cyclin-dependent kinase 1 | Homininae | CDK1_HUMAN | P06493 | 34 kDa |
| Cyclin-dependent kinase 12 | Hominidae | CDK12_HUMAN | Q9NYV4 | 164 kDa |
| Cysteine and glycine-rich | Homo sapiens | B3KVC9_HUMAN | B3KVC9 | 18 kDa |
| protein 1 | | | (+1) | |
| Cytochrome b-c1 complex | Homo sapiens | QCR2_HUMAN | P22695 | 48 kDa |
| subunit 2, mitochondrial | | | | |
| Cytochrome c | Hominoidea | CYC_HUMAN | P99999 | 12 kDa |
| Cytochrome c oxidase | Homo sapiens | A0S0W7_HUMAN | A0S0W7 | 26 kDa |
| subunit 2 | | | (+80) | |
| Cytoplasmic aconitate | Homo sapiens | ACOC_HUMAN | P21399 | 98 kDa |
| hydratase | | | | |
| Cytoplasmic dynein 1 heavy | Eutheria | DYHC1_HUMAN | Q14204 | 532 kDa |
| chain 1 | | | | |
| Death-inducer obliterator 1 | Homo sapiens | DIDO1_HUMAN | Q9BTC0 | 244 kDa |
| Delta(3,5)-Delta(2,4)- | Homo sapiens | ECH1_HUMAN | Q13011 | 36 kDa |
| dienoyl-CoA isomerase, | | | | |
| mitochondrial | | | | |
| Desmoglein-2 | Homo sapiens | DSG2_HUMAN | Q14126 | 122 kDa |
| Desmoplakin | Homo sapiens | DESP_HUMAN | P15924 | 332 kDa |
| Developmentally-regulated | Eutheria | DRG1_HUMAN | Q9Y295 | 41 kDa |
| GTP-binding protein 1 | | | | |
| Dihydrolipoyl | Homininae | DLDH_HUMAN | P09622 | 54 kDa |
| dehydrogenase, | | | | |
| mitochondrial | | | | |
| Dihydrolipoyllysine-residue | Homo sapiens | ODP2_HUMAN | P10515 | 69 kDa |
| acetyltransferase component | | | | |
| of pyruvate dehydrogenase | | | | |
| complex, mitochondrial | | | | |
| Dihydropyrimidinase-related | Catarrhini | DPYL2_HUMAN | Q16555 | 62 kDa |
| protein 2 | | | | |
| Dipeptidyl peptidase 3 | Homo sapiens | DPP3_HUMAN | Q9NY33 | 83 kDa |
| DNA damage-binding protein | Simiiformes | DDB1_HUMAN | Q16531 | 127 kDa |
| 1 | | | | |
| DNA replication licensing | Hominidae | MCM2_HUMAN | P49736 | 102 kDa |
| factor MCM2 | | | | |
| DNA replication licensing | Homininae | MCM3_HUMAN | P25205 | 91 kDa |
| factor MCM3 | | | | |
| DNA replication licensing | Homo sapiens | MCM4_HUMAN | P33991 | 97 kDa |
| factor MCM4 | | | | |
| DNA replication licensing | Homo sapiens | MCM5_HUMAN | P33992 | 82 kDa |
| factor MCM5 | | | | |
| DNA-(apurinic or | Homo sapiens | APEX1_HUMAN | P27695 | 36 kDa |
| apyrimidinic site) lyase | | | | |
| DNA-binding protein A | Homo sapiens | DBPA_HUMAN | P16989 | 40 kDa |
| | | | (+1) | |
| DNA-binding protein Ikaros | Catarrhini | IKZF1_HUMAN | Q13422 | 58 kDa |
| DNA-dependent protein | Homo sapiens | PRKDC_HUMAN | P78527 | 469 kDa |
| kinase catalytic subunit | | | | |
| DnaJ homolog subfamily A | Eutheria | DNJA1_HUMAN | P31689 | 45 kDa |
| member 1 | | | | |
| DnaJ homolog subfamily B | Hominidae | DNJB1_HUMAN | P25685 | 38 kDa |
| member 1 | | | (+1) | |
| DnaJ homolog subfamily C | Homo sapiens | DJC13_HUMAN | O75165 | 254 kDa |
| member 13 | | | | |
| Dolichyl- | Homo sapiens | RPN1_HUMAN | P04843 | 69 kDa |
| diphosphooligosaccharide-- | | | (+1) | |
| protein glycosyltransferase | | | | |
| subunit 1 | | | | |
| Dolichyl- | Homo sapiens | RPN2_HUMAN | P04844 | 69 kDa |
| diphosphooligosaccharide-- | | | | |
| protein glycosyltransferase | | | | |
| subunit 2 | | | | |
| Dopamine beta-hydroxylase | Homo sapiens | DOPO_HUMAN | P09172 | 69 kDa |
| Dopamine receptor | Homo sapiens | Q4W4Y1_HUMAN | Q4W4Y1 | 96 kDa |
| interacting protein 4 | | | (+2) | |
| Double-stranded RNA- | Homo sapiens | DSRAD_HUMAN | P55265 | 136 kDa |
| specific adenosine deaminase | | | (+1) | |
| DPYSL3 protein | Homo sapiens | Q6DEN2_HUMAN | Q6DEN2 | 74 kDa |
| Dual oxidase 2 | Homo sapiens | DUOX2_HUMAN | Q9NRD8 | 175 kDa |
| Dynactin subunit 2 | Homo sapiens | DCTN2_HUMAN | Q13561 | 44 kDa |
| Dynamin-1-like protein | Catarrhini | DNM1L_HUMAN | O00429 | 82 kDa |
| Dynein heavy chain 1, | Homo sapiens | DYH1_HUMAN | Q9P2D7 | 494 kDa |
| axonemal | | | | |
| Dynein heavy chain 2, | Homo sapiens | DYH2_HUMAN | Q9P225 | 508 kDa |
| axonemal | | | | |
| Dystonin | Homo sapiens | DYST_HUMAN | Q03001 | 861 kDa |
| E3 ubiquitm/ISG15 ligase | Homo sapiens | TRI25_HUMAN | Q14258 | 71 kDa |
| TRIM25 | | | | |
| E3 ubiquitin-protein ligase | Homo sapiens | HUWE1_HUMAN | Q7Z6Z7 | 482 kDa |
| HUWE1 | | | | |
| E3 ubiquitin-protein ligase | Homo sapiens | UBR4_HUMAN | Q5T4S7 | 574 kDa |
| UBR4 | | | | |
| Early endosome antigen 1 | Homo sapiens | EEA1_HUMAN | Q15075 | 162 kDa |
| EF-hand domain-containing | Homo sapiens | EFHD2_HUMAN | Q96C19 | 27 kDa |
| protein D2 | | | | |
| Electron transfer flavoprotein | Homo sapiens | ETFA_HUMAN | P13804 | 35 kDa |
| subunit alpha, mitochondrial | | | | |
| Elongation factor 1-alpha 1 | Eutheria | EF1A1_HUMAN | P68104 | 50 kDa |
| | | | (+1) | |
| Elongation factor 1-delta | Homo sapiens | EF1D_HUMAN | P29692 | 31 kDa |
| Elongation factor 1-gamma | Homo sapiens | EF1G_HUMAN | P26641 | 50 kDa |
| Elongation factor 2 | Hominidae | EF2_HUMAN | P13639 | 95 kDa |
| Elongation factor Tu, | Homininae | EFTU_HUMAN | P49411 | 50 kDa |
| mitochondrial | | | | |
| Endoplasmic reticulum | Homo sapiens | ERAP1_HUMAN | Q9NZ08 | 107 kDa |
| aminopeptidase 1 | | | | |
| Endoplasmin | Homo sapiens | ENPL_HUMAN | P14625 | 92 kDa |
| | | | (+1) | |
| Enoyl-CoA hydratase, | Homo sapiens | ECHM_HUMAN | P30084 | 31 kDa |
| mitochondrial | | | | |
| Ephrin type-A receptor 2 | Homo sapiens | EPHA2_HUMAN | P29317 | 108 kDa |
| ERBB2IP protein | Homo sapiens | Q1RMC9_HUMAN | Q1RMC9 | 154 kDa |
| | | | (+2) | |
| Erlin-1 | Homininae | ERLN1_HUMAN | O75477 | 39 kDa |
| Erlin-2 | Hominidae | ERLN2_HUMAN | O94905 | 38 kDa |
| Eukaryotic initiation factor | Eutheria | IF4A1_HUMAN | P60842 | 46 kDa |
| 4A-I | | | | |
| Eukaryotic translation | Catarrhini | IF2A_HUMAN | P05198 | 36 kDa |
| initiation factor 2 subunit 1 | | | | |
| Eukaryotic translation | Homo sapiens | EIF3A_HUMAN | Q14152 | 167 kDa |
| initiation factor 3 subunit A | | | (+1) | |
| Eukaryotic translation | Homo sapiens | EIF3B_HUMAN | P55884 | 92 kDa |
| initiation factor 3 subunit B | | | | |
| Eukaryotic translation | Eutheria | EIF3M_HUMAN | Q7L2H7 | 43 kDa |
| initiation factor 3 subunit M | | | | |
| Eukaryotic translation | Homo sapiens | IF4G1_HUMAN | Q04637 | 175 kDa |
| initiation factor 4 gamma 1 | | | | |
| Eukaryotic translation | Homininae | IF4H_HUMAN | Q15056 | 27 kDa |
| initiation factor 4H | | | | |
| Eukaryotic translation | Homo sapiens | IF5_HUMAN | P55010 | 49 kDa |
| initiation factor 5 | | | (+1) | |
| Eukaryotic translation | Tetrapoda | IF5A1_HUMAN | P63241 | 17 kDa |
| initiation factor 5A-1 | | | (+1) | |
| Eukaryotic translation | Catarrhini | IF6_HUMAN | P56537 | 27 kDa |
| initiation factor 6 | | | | |
| Exportin-1 | Simiiformes | XPO1_HUMAN | O14980 | 123 kDa |
| Exportin-7 | Homininae | XPO7_HUMAN | Q9UIA9 | 124 kDa |
| Exportin-T | Homininae | XPOT_HUMAN | O43592 | 110 kDa |
| Extended synaptotagmin-1 | Homo sapiens | ESYT1_HUMAN | Q9BSJ8 | 123 kDa |
| Extended synaptotagmin-2 | Homo sapiens | ESYT2_HUMAN | A0FGR8 | 102 kDa |
| F-actin-capping protein | Homininae | CAZA1_HUMAN | P52907 | 33 kDa |
| subunit alpha-1 | | | | |
| F-actin-capping protein | Hominidae | CAPZB_HUMAN | P47756 | 31 kDa |
| subunit beta | | | | |
| Far upstream element- | Homo sapiens | FUBP2_HUMAN | Q92945 | 73 kDa |
| binding protein 2 | | | | |
| Farnesyl pyrophosphate | Homininae | FPPS_HUMAN | P14324 | 48 kDa |
| synthase | | | | |
| FAS-associated factor 2 | Homo sapiens | FAF2_HUMAN | Q96CS3 | 53 kDa |
| Fascin | Homininae | FSCN1_HUMAN | Q16658 | 55 kDa |
| Fatty acid synthase | Homo sapiens | FAS_HUMAN | P49327 | 273 kDa |
| Fermitin family homolog 3 | Homininae | URP2_HUMAN | Q86UX7 | 76 kDa |
| Fibrillin-1 | Homo sapiens | FBN1_HUMAN | P35555 | 312 kDa |
| Fibrinogen alpha chain | Homo sapiens | FIBA_HUMAN | P02671 | 95 kDa |
| Fibrinogen beta chain | Homo sapiens | FIBB_HUMAN | P02675 | 56 kDa |
| Fibrinogen gamma chain | Homo sapiens | FIBG_HUMAN | P02679 | 52 kDa |
| Fibronectin | Homo sapiens | FINC_HUMAN | P02751 | 263 kDa |
| Filamin B | Homo sapiens | B2ZZ83_HUMAN | B2ZZ83 | 282 kDa |
| | | | (+1) | |
| Filamin-A | Homo sapiens | FLNA_HUMAN | P21333 | 281 kDa |
| | | | (+1) | |
| Filamin-C | Homo sapiens | FLNC_HUMAN | Q14315 | 291 kDa |
| Flotillin-1 | Homo sapiens | FLOT1_HUMAN | O75955 | 47 kDa |
| | | | (+1) | |
| Fructose-bisphosphate | Hominoidea | ALDOA_HUMAN | P04075 | 39 kDa |
| aldolase A | | | | |
| Fructose-bisphosphate | Homininae | ALDOC_HUMAN | P09972 | 39 kDa |
| aldolase C | | | | |
| Fumarate hydratase, | Homo sapiens | FUMH_HUMAN | P07954 | 55 kDa |
| mitochondrial | | | | |
| G patch domain-containing | Homo sapiens | GPTC8_HUMAN | Q9UKJ3 | 164 kDa |
| protein 8 | | | | |
| Galectin-1 | Homininae | LEG1_HUMAN | P09382 | 15 kDa |
| Gelsolin | Homo sapiens | GELS_HUMAN | P06396 | 86 kDa |
| Glucosamine 6-phosphate N- | Eutheria | GNA1_HUMAN | Q96EK6 | 21 kDa |
| acetyltransferase | | | | |
| Glucosamine--fructose-6- | Homininae | GFPT1_HUMAN | Q06210 | 79 kDa |
| phosphate aminotransferase | | | | |
| [isomerizing] 1 | | | | |
| Glucose-6-phosphate 1- | Homininae | G6PD_HUMAN | P11413 | 59 kDa |
| dehydrogenase | | | | |
| Glucose-6-phosphate | Homo sapiens | G6PI_HUMAN | P06744 | 63 kDa |
| isomerase | | | | |
| Glucosidase 2 subunit beta | Homo sapiens | GLU2B_HUMAN | P14314 | 59 kDa |
| Glutamate dehydrogenase 1, | Homo sapiens | DHE3_HUMAN | P00367 | 61 kDa |
| mitochondrial | | | | |
| Glutamate--cysteine ligase | Homo sapiens | GSH1_HUMAN | P48506 | 73 kDa |
| catalytic subunit | | | | |
| Glutaminase kidney isoform, | Homininae | GLSK_HUMAN | O94925 | 73 kDa |
| mitochondrial | | | | |
| Glutamine--tRNA ligase | Homo sapiens | SYQ_HUMAN | P47897 | 88 kDa |
| Glutathione S-transferase | Homo sapiens | GSTO1_HUMAN | P78417 | 28 kDa |
| omega-1 | | | | |
| Glutathione S-transferase P | Homo sapiens | GSTP1_HUMAN | P09211 | 23 kDa |
| Glyceraldehyde-3-phosphate | Homininae | G3P_HUMAN | P04406 | 36 kDa |
| dehydrogenase | | | | |
| Glycine--tRNA ligase | Homo sapiens | SYG_HUMAN | P41250 | 83 kDa |
| Glycogen phosphorylase, | Homo sapiens | PYGB_HUMAN | P11216 | 97 kDa |
| brain form | | | | |
| Glycogen phosphorylase, | Homininae | PYGL_HUMAN | P06737 | 97 kDa |
| liver form | | | | |
| Golgi apparatus protein 1 | Homo sapiens | GSLG1_HUMAN | Q92896 | 135 kDa |
| Guanine nucleotide-binding | Tetrapoda | GBLP_HUMAN | P63244 | 35 kDa |
| protein subunit beta-2-like 1 | | | | |
| Haptoglobin | Homo sapiens | HPT_HUMAN | P00738 | 45 kDa |
| HEAT repeat-containing | Homo sapiens | HTR5A_HUMAN | Q86XA9 | 222 kDa |
| protein 5A | | | | |
| Heat shock 70 kDa protein | Catarrhini | HSP71_HUMAN | P08107 | 70 kDa |
| 1A/1B | | | | |
| Heat shock 70 kDa protein 4 | Homo sapiens | HSP74_HUMAN | P34932 | 94 kDa |
| Heat shock cognate 71 kDa | Eutheria | HSP7C_HUMAN | P11142 | 71 kDa |
| protein | | | | |
| Heat shock protein 105 kDa | Hominidae | HS105_HUMAN | Q92598 | 97 kDa |
| Heat shock protein beta-1 | Catarrhini | HSPB1_HUMAN | P04792 | 23 kDa |
| Heat shock protein HSP 90- | Simiiformes | HS90A_HUMAN | P07900 | 85 kDa |
| alpha | | | | |
| Heat shock protein HSP 90- | Homo sapiens | HS90B_HUMAN | P08238 | 83 kDa |
| beta | | | | |
| Hemoglobin subunit alpha | Hominidae | HBA_HUMAN | P69905 | 15 kDa |
| Hemoglobin subunit beta | Homininae | HBB_HUMAN | P68871 | 16 kDa |
| Hemoglobin subunit delta | Homo sapiens | HBD_HUMAN | P02042 | 16 kDa |
| Hemoglobin subunit gamma-2 | Homininae | HBG2_HUMAN | P69892 | 16 kDa |
| Hemopexin | Homo sapiens | HEMO_HUMAN | P02790 | 52 kDa |
| Heparin cofactor 2 | Homo sapiens | HEP2_HUMAN | P05546 | 57 kDa |
| Hepatoma-derived growth | Homo sapiens | HDGF_HUMAN | P51858 | 27 kDa |
| factor | | | | |
| Hepatoma-derived growth | Homo sapiens | HDGR2_HUMAN | Q7Z4V5 | 74 kDa |
| factor-related protein 2 | | | | |
| Hepatopoietin PCn127 | Homo sapiens | Q1AHP8_HUMAN | Q1AHP8 | 28 kDa |
| | | | (+5) | |
| Heterogeneous nuclear | Homo sapiens | ROAA_HUMAN | Q99729 | 36 kDa |
| ribonucleoprotein A/B | | | | |
| Heterogeneous nuclear | Homo sapiens | ROA1_HUMAN | P09651 | 39 kDa |
| ribonucleoprotein A1 | | | | |
| Heterogeneous nuclear | Eutheria | HNRH1_HUMAN | P31943 | 49 kDa |
| ribonucleoprotein H | | | | |
| Heterogeneous nuclear | Eutheria | HNRPK_HUMAN | P61978 | 51 kDa |
| ribonucleoprotein K | | | (+1) | |
| Heterogeneous nuclear | Eutheria | HNRPQ_HUMAN | O60506 | 70 kDa |
| ribonucleoprotein Q | | | | |
| Heterogeneous nuclear | Eutheria | HNRPR_HUMAN | O43390 | 71 kDa |
| ribonucleoprotein R | | | (+1) | |
| Heterogeneous nuclear | Homo sapiens | HNRPU_HUMAN | Q00839 | 91 kDa |
| ribonucleoprotein U | | | | |
| Heterogeneous nuclear | Eutheria | ROA2_HUMAN | P22626 | 37 kDa |
| ribonucleoproteins A2/B1 | | | | |
| Heterogeneous nuclear | Catarrhini | HNRPC_HUMAN | P07910 | 34 kDa |
| ribonucleoproteins C1/C2 | | | | |
| Hippocalcin-like protein 1 | Homo sapiens | HPCL1_HUMAN | P37235 | 22 kDa |
| Histidine-rich glycoprotein | Homo sapiens | HRG_HUMAN | P04196 | 60 kDa |
| HMG box transcription factor | Homo sapiens | BBX_HUMAN | Q8WY36 | 105 kDa |
| BBX | | | | |
| Hsp70-binding protein 1 | Homo sapiens | HPBP1_HUMAN | Q9NZL4 | 39 kDa |
| Hydroxymethylglutaryl-CoA | Homo sapiens | HMCS1_HUMAN | Q01581 | 57 kDa |
| synthase, cytoplasmic | | | (+1) | |
| Hypoxanthine-guanine | Catarrhini | HPRT_HUMAN | P00492 | 25 kDa |
| phosphoribosyltransferase | | | | |
| Hypoxia up-regulated protein | Homo sapiens | HYOU1_HUMAN | Q9Y4L1 | 111 kDa |
| 1 | | | | |
| Ig alpha-1 chain C region | Homo sapiens | IGHA1_HUMAN | P01876 | 38 kDa |
| Ig gamma-2 chain C region | Homo sapiens | IGHG2_HUMAN | P01859 | 36 kDa |
| Ig gamma-4 chain C region | Homo sapiens | IGHG4_HUMAN | P01861 | 36 kDa |
| Ig heavy chain V-I region | Homo sapiens | HV102_HUMAN | P01743 | 13 kDa |
| HG3 | | | | |
| Ig kappa chain V-I region | Homo sapiens | KV102_HUMAN | P01594 | 12 kDa |
| AU | | | | |
| Ig kappa chain V-II region | Homo sapiens | KV204_HUMAN | P01617 | 12 kDa |
| TEW | | | | |
| Ig kappa chain V-III region | Homo sapiens | KV304_HUMAN | P01622 | 12 kDa |
| Ti | | | | |
| Ig mu chain C region | Homo sapiens | IGHM_HUMAN | P01871 | 49 kDa |
| IGH@ protein | Homo sapiens | Q6GMX6_HUMAN | Q6GMX6 | 51 kDa |
| IGK@ protein | Homo sapiens | Q6PIL8_HUMAN | Q6PIL8 | 26 kDa |
| IGK@ protein | Homo sapiens | Q6P5S8_HUMAN | Q6P5S8 | 26 kDa |
| IGL@ protein | Homo sapiens | Q567P1_HUMAN | Q567P1 | 25 kDa |
| Immunoglobulin J chain | Homo sapiens | IGJ_HUMAN | P01591 | 18 kDa |
| Importin subunit alpha-2 | Homo sapiens | IMA2_HUMAN | P52292 | 58 kDa |
| | | | (+1) | |
| Importin subunit beta-1 | Hominoidea | IMB1_HUMAN | Q14974 | 97 kDa |
| Importin-4 | Homo sapiens | IPO4_HUMAN | Q8TEX9 | 119 kDa |
| Importin-5 | Homo sapiens | IPO5_HUMAN | O00410 | 124 kDa |
| Importin-7 | Simiiformes | IPO7_HUMAN | O95373 | 120 kDa |
| Inositol-3-phosphate synthase | Homo sapiens | INO1_HUMAN | Q9NPH2 | 61 kDa |
| 1 | | | | |
| Insulin-like growth factor 2 | Homininae | IF2B2_HUMAN | Q9Y6M1 | 66 kDa |
| mRNA-binding protein 2 | | | | |
| Insulin-like growth factor- | Homo sapiens | ALS_HUMAN | P35858 | 66 kDa |
| binding protein complex acid | | | (+1) | |
| labile subunit | | | | |
| Integrin beta-1 | Homininae | ITB1_HUMAN | P05556 | 88 kDa |
| Integrin beta-4 | Homo sapiens | ITB4_HUMAN | P16144 | 202 kDa |
| Inter-alpha (Globulin) | Homo sapiens | B2RMS9_HUMAN | B2RMS9 | 103 kDa |
| inhibitor H4 (Plasma | | | (+2) | |
| Kallikrein-sensitive | | | | |
| glycoprotein) | | | | |
| Inter-alpha-trypsin inhibitor | Homo sapiens | ITIH1_HUMAN | P19827 | 101 kDa |
| heavy chain H1 | | | | |
| Inter-alpha-trypsin inhibitor | Homo sapiens | ITIH2_HUMAN | P19823 | 106 kDa |
| heavy chain H2 | | | (+1) | |
| Intercellular adhesion | Homo sapiens | ICAM1_HUMAN | P05362 | 58 kDa |
| molecule 1 | | | | |
| Interleukin enhancer-binding | Eutheria | ILF2_HUMAN | Q12905 | 43 kDa |
| factor 2 | | | | |
| Interleukin enhancer-binding | Homo sapiens | ILF3_HUMAN | Q12906 | 95 kDa |
| factor 3 | | | | |
| Iron-responsive element- | Homo sapiens | IREB2_HUMAN | P48200 | 105 kDa |
| binding protein 2 | | | | |
| Iron-sulfur protein NUBPL | Homo sapiens | NUBPL_HUMAN | Q8TB37 | 34 kDa |
| Isocitrate dehydrogenase | Homo sapiens | IDHC_HUMAN | O75874 | 47 kDa |
| [NADP] cytoplasmic | | | | |
| Junction plakoglobin | Homininae | PLAK_HUMAN | P14923 | 82 kDa |
| Kinesin-1 heavy chain | Homo sapiens | KINH_HUMAN | P33176 | 110 kDa |
| Kinesin-associated protein 3 | Homininae | KIFA3_HUMAN | Q92845 | 91 kDa |
| Kininogen-1 | Homo sapiens | KNG1_HUMAN | P01042 | 72 kDa |
| | | | (+1) | |
| Kynurenine/alpha- | Catarrhini | AADAT_HUMAN | Q8N5Z0 | 47 kDa |
| aminoadipate | | | | |
| aminotransferase, | | | | |
| mitochondrial | | | | |
| Lactoylglutathione lyase | Homo sapiens | LGUL_HUMAN | Q04760 | 21 kDa |
| LanC-like protein 2 | Homo sapiens | LANC2_HUMAN | Q9NS86 | 51 kDa |
| Large neutral amino acids | Homo sapiens | LAT1_HUMAN | Q01650 | 55 kDa |
| transporter small subunit 1 | | | (+1) | |
| Large proline-rich protein | Homo sapiens | BAG6_HUMAN | P46379 | 119 kDa |
| BAG6 | | | | |
| Leucine-rich alpha-2- | Homo sapiens | A2GL_HUMAN | P02750 | 38 kDa |
| glycoprotein | | | | |
| Leucine-rich PPR motif- | Homo sapiens | LPPRC_HUMAN | P42704 | 158 kDa |
| containing protein, | | | | |
| mitochondrial | | | | |
| Leucine-rich repeat | Homo sapiens | LRRF1_HUMAN | Q32MZ4 | 89 kDa |
| flightless-interacting protein | | | | |
| 1 | | | | |
| Leucine--tRNA ligase, | Homo sapiens | SYLC_HUMAN | Q9P2J5 | 134 kDa |
| cytoplasmic | | | | |
| Leukotriene A-4 hydrolase | Homininae | LKHA4_HUMAN | P09960 | 69 kDa |
| Liprin-alpha-1 | Homo sapiens | LIPA1_HUMAN | Q13136 | 136 kDa |
| L-lactate dehydrogenase A | Homo sapiens | LDHA_HUMAN | P00338 | 37 kDa |
| chain | | | | |
| L-lactate dehydrogenase B | Catarrhini | LDHB_HUMAN | P07195 | 37 kDa |
| chain | | | | |
| Lysine--tRNA ligase | Homo sapiens | SYK_HUMAN | Q15046 | 68 kDa |
| Lysophosphatidylcholine | Homo sapiens | PCAT1_HUMAN | Q8NF37 | 59 kDa |
| acyltransferase 1 | | | | |
| Macrophage mannose | Homo sapiens | MRC1_HUMAN | P22897 | 166 kDa |
| receptor 1 | | | (+1) | |
| Macrophage-capping protein | Homo sapiens | CAPG_HUMAN | P40121 | 38 kDa |
| Major vault protein | Homo sapiens | MVP_HUMAN | Q14764 | 99 kDa |
| Malate dehydrogenase, | Homo sapiens | MDHC_HUMAN | P40925 | 36 kDa |
| cytoplasmic | | | | |
| Malate dehydrogenase, | Homo sapiens | MDHM_HUMAN | P40926 | 36 kDa |
| mitochondrial | | | (+1) | |
| Mannosyl-oligosaccharide | Homo sapiens | MOGS_HUMAN | Q13724 | 92 kDa |
| glucosidase | | | (+1) | |
| Microsomal glutathione S- | Homininae | MGST1_HUMAN | P10620 | 18 kDa |
| transferase 1 | | | | |
| Microtubule-actin cross- | Homo sapiens | MACF1_HUMAN | Q9UPN3 | 838 kDa |
| linking factor 1, isoforms | | | | |
| 1/2/3/5 | | | | |
| Microtubule-associated | Homininae | MAP1B_HUMAN | P46821 | 271 kDa |
| protein 1B | | | | |
| Microtubule-associated | Homo sapiens | MAP4_HUMAN | P27816 | 121 kDa |
| protein 4 | | | | |
| MKI67 FHA domain- | Homo sapiens | MK67I_HUMAN | Q9BYG3 | 34 kDa |
| interacting nucleolar | | | | |
| phosphoprotein | | | | |
| Moesin | Catarrhini | MOES_HUMAN | P26038 | 68 kDa |
| Monofunctional C1- | Homo sapiens | C1TM_HUMAN | Q6UB35 | 106 kDa |
| tetrahydrofolate synthase, | | | | |
| mitochondrial | | | | |
| Msx2-interacting protein | Homo sapiens | MINT_HUMAN | Q96T58 | 402 kDa |
| Multifunctional protein | Homo sapiens | PUR6_HUMAN | P22234 | 47 kDa |
| ADE2 | | | | |
| Myb-binding protein 1A | Homo sapiens | MBB1A_HUMAN | Q9BQG0 | 149 kDa |
| Myoferlin | Homo sapiens | MYOF_HUMAN | Q9NZM1 | 235 kDa |
| Myosin-10 | Homininae | MYH10_HUMAN | P35580 | 229 kDa |
| Myosin-6 | Homo sapiens | MYH6_HUMAN | P13533 | 224 kDa |
| Myosin-9 | Homo sapiens | MYH9_HUMAN | P35579 | 227 kDa |
| N-acetylmuramoyl-L-alanine | Homo sapiens | PGRP2_HUMAN | Q96PD5 | 62 kDa |
| amidase | | | | |
| NACHT, LRR and PYD | Homo sapiens | NALP2_HUMAN | Q9NX02 | 121 kDa |
| domains-containing protein 2 | | | | |
| NAD(P)H dehydrogenase | Homininae | NQO1_HUMAN | P15559 | 31 kDa |
| [quinone] 1 | | | | |
| NAD(P)H-hydrate epimerase | Homo sapiens | NNRE_HUMAN | Q8NCW5 | 32 kDa |
| NADH-cytochrome b5 | Homo sapiens | NB5R3_HUMAN | P00387 | 34 kDa |
| reductase 3 | | | | |
| NADPH--cytochrome P450 | Homo sapiens | NCPR_HUMAN | P16435 | 77 kDa |
| reductase | | | | |
| Nascent polypeptide- | Theria | NACA_HUMAN | Q13765 | 23 kDa |
| associated complex subunit | | | | |
| alpha | | | | |
| Nebulin | Homo sapiens | NEBU_HUMAN | P20929 | 773 kDa |
| Nesprin-2 | Homo sapiens | SYNE2_HUMAN | Q8WXH0 | 796 kDa |
| Neuroblast differentiation- | Homo sapiens | AHNK_HUMAN | Q09666 | 629 kDa |
| associated protein AHNAK | | | | |
| Neurofibromin | Catarrhini | NF1_HUMAN | P21359 | 319 kDa |
| Neutral alpha-glucosidase | Homo sapiens | GANAB_HUMAN | Q14697 | 107 kDa |
| AB | | | | |
| Niban-like protein 2 | Homo sapiens | NIBL2_HUMAN | Q86XR2 | 77 kDa |
| Nicotinamide | Homininae | NAMPT_HUMAN | P43490 | 56 kDa |
| phosphoribosyltransferase | | | | |
| Nitrilase homolog 1 | Homo sapiens | NIT1_HUMAN | Q86X76 | 36 kDa |
| N-lysine methyltransferase | Homo sapiens | SETD6_HUMAN | Q8TBK2 | 53 kDa |
| SETD6 | | | | |
| Nodal modulator 3 | Homo sapiens | NOMO3_HUMAN | P69849 | 134 kDa |
| | | | (+3) | |
| Nuclear factor erythroid 2- | Homo sapiens | NF2L3_HUMAN | Q9Y4A8 | 76 kDa |
| related factor 3 | | | (+1) | |
| Nuclear migration protein | Homo sapiens | NUDC_HUMAN | Q9Y266 | 38 kDa |
| nudC | | | | |
| Nucleolar GTP-binding | Homo sapiens | NOG2_HUMAN | Q13823 | 84 kDa |
| protein 2 | | | | |
| Nucleolar RNA helicase 2 | Homo sapiens | DDX21_HUMAN | Q9NR30 | 87 kDa |
| Nucleolin | Homo sapiens | NUCL_HUMAN | P19338 | 77 kDa |
| Nucleophosmin | Homininae | NPM_HUMAN | P06748 | 33 kDa |
| Nucleoside diphosphate | Hominidae | NDKA_HUMAN | P15531 | 17 kDa |
| kinase A | | | | |
| Nucleosome assembly | Homininae | NP1L1_HUMAN | P55209 | 45 kDa |
| protein 1-like 1 | | | | |
| Obg-like ATPase 1 | Eutheria | OLA1_HUMAN | Q9NTK5 | 45 kDa |
| Ornithine aminotransferase, | Homininae | OAT_HUMAN | P04181 | 49 kDa |
| mitochondrial | | | | |
| Oxysterol-binding protein 1 | Homo sapiens | OSBP1_HUMAN | P22059 | 89 kDa |
| Pachytene checkpoint protein | Homo sapiens | PCH2_HUMAN | Q15645 | 49 kDa |
| 2 homolog | | | | |
| Peptidyl-prolyl cis-trans | Catarrhini | Q567Q0_HUMAN | Q567Q0 | 11 kDa |
| isomerase A | | | | |
| Peptidyl-prolyl cis-trans | Catarrhini | PPIB_HUMAN | P23284 | 24 kDa |
| isomerase B | | | | |
| Peroxiredoxin-1 | Homininae | PRDX1_HUMAN | Q06830 | 22 kDa |
| Peroxiredoxin-2 | Catarrhini | PRDX2_HUMAN | P32119 | 22 kDa |
| Peroxiredoxin-5, | Homo sapiens | PRDX5_HUMAN | P30044 | 22 kDa |
| mitochondrial | | | | |
| Peroxiredoxin-6 | Hominidae | PRDX6_HUMAN | P30041 | 25 kDa |
| Peroxisomal multifunctional | Homo sapiens | DHB4_HUMAN | P51659 | 80 kDa |
| enzyme type 2 | | | | |
| Phosphatidylinositol-binding | Homo sapiens | PICAL_HUMAN | Q13492 | 71 kDa |
| clathrin assembly protein | | | | |
| Phosphatidylinositol-glycan- | Homo sapiens | PHLD_HUMAN | P80108 | 92 kDa |
| specific phospholipase D | | | | |
| Phosphoglucomutase-1 | Homo sapiens | PGM1_HUMAN | P36871 | 61 kDa |
| Phosphoglycerate kinase 1 | Euarchontoglires | PGK1_HUMAN | P00558 | 45 kDa |
| Phosphoglycerate mutase 1 | Eutheria | PGAM1_HUMAN | P18669 | 29 kDa |
| Phosphoribosylglycinamide | Homo sapiens | Q3B7A7_HUMAN | Q3B7A7 | 108 kDa |
| formyltransferase, | | | (+1) | |
| phosphoribosylglycinamide | | | | |
| synthetase, | | | | |
| phosphoribosylaminoimidazole | | | | |
| synthetase | | | | |
| Phosphoserine | Homo sapiens | SERC_HUMAN | Q9Y617 | 40 kDa |
| aminotransferase | | | | |
| PIG48 | Homo sapiens | Q2TU64_HUMAN | Q2TU64 | 61 kDa |
| Pigment epithelium-derived | Homo sapiens | PEDF_HUMAN | P36955 | 46 kDa |
| factor | | | | |
| Plasma kallikrein | Homo sapiens | KLKB1_HUMAN | P03952 | 71 kDa |
| Plasma protease C1 inhibitor | Homo sapiens | IC1_HUMAN | P05155 | 55 kDa |
| Plasminogen | Homo sapiens | PLMN_HUMAN | P00747 | 91 kDa |
| Plasminogen activator | Hominoidea | PAIRB_HUMAN | Q8NC51 | 45 kDa |
| inhibitor 1 RNA-binding | | | | |
| protein | | | | |
| Platelet-activating factor | Homininae | PA1B3_HUMAN | Q15102 | 26 kDa |
| acetylhydrolase IB subunit | | | | |
| gamma | | | | |
| Plectin | Homo sapiens | PLEC_HUMAN | Q15149 | 532 kDa |
| Poly [ADP-ribose] | Homo sapiens | PARP1_HUMAN | P09874 | 113 kDa |
| polymerase 1 | | | | |
| Poly(RC) binding protein 2 | Euarchontoglires | Q68Y55_HUMAN | Q68Y55 | 35 kDa |
| Poly(rC)-binding protein 1 | Eutheria | PCBP1_HUMAN | Q15365 | 37 kDa |
| Polyadenylate-binding | Eutheria | PABP1_HUMAN | P11940 | 71 kDa |
| protein 1 | | | | |
| Polyadenylate-binding | Homo sapiens | PABP4_HUMAN | Q13310 | 71 kDa |
| protein 4 | | | (+1) | |
| Polymerase I and transcript | Homo sapiens | PTRF_HUMAN | Q6NZI2 | 43 kDa |
| release factor | | | | |
| Prelamin-A/C | Homininae | LMNA_HUMAN | P02545 | 74 kDa |
| | | | (+3) | |
| Pre-mRNA-processing factor | Homo sapiens | PR40A_HUMAN | O75400 | 109 kDa |
| 40 homolog A | | | | |
| Prenylcysteine oxidase 1 | Homo sapiens | PCYOX_HUMAN | Q9UHG3 | 57 kDa |
| Probable ATP-dependent | Hominidae | DDX17_HUMAN | Q92841 | 80 kDa |
| RNA helicase DDX17 | | | | |
| Probable ATP-dependent | Hominoidea | DDX6_HUMAN | P26196 | 54 kDa |
| RNA helicase DDX6 | | | | |
| Probable E3 ubiquitin-protein | Homo sapiens | HERC1_HUMAN | Q15751 | 532 kDa |
| ligase HERC1 | | | | |
| Probable phospholipid- | Homo sapiens | AT10D_HUMAN | Q9P241 | 160 kDa |
| transporting ATPase VD | | | | |
| Probable RNA-binding | Homo sapiens | RBM20_HUMAN | Q5T481 | 134 kDa |
| protein 20 | | | | |
| Profilin-1 | Catarrhini | PROF1_HUMAN | P07737 | 15 kDa |
| Programmed cell death | Homo sapiens | PDCD4_HUMAN | Q53EL6 | 52 kDa |
| protein 4 | | | | |
| Prohibitin | Eutheria | PHB_HUMAN | P35232 | 30 kDa |
| Prohibitin-2 | Euarchontoglires | PHB2_HUMAN | Q99623 | 33 kDa |
| Proliferating cell nuclear | Homo sapiens | Q6FI35_HUMAN | Q6FI35 | 29 kDa |
| antigen | | | | |
| Proline-, glutamic acid- and | Homo sapiens | PELP1_HUMAN | Q8IZL8 | 120 kDa |
| leucine-rich protein 1 | | | | |
| Prolyl 3-hydroxylase 1 | Homo sapiens | P3H1_HUMAN | Q32P28 | 83 kDa |
| Prolyl endopeptidase | Homo sapiens | PPCE_HUMAN | P48147 | 81 kDa |
| Proteasome activator | Hominidae | PSME1_HUMAN | Q06323 | 29 kDa |
| complex subunit 1 | | | | |
| Proteasome activator | Eutheria | PSME3_HUMAN | P61289 | 30 kDa |
| complex subunit 3 | | | | |
| Proteasome subunit alpha | Eutheria | PSA1_HUMAN | P25786 | 30 kDa |
| type-1 | | | | |
| Proteasome subunit alpha | Eutheria | PSA3_HUMAN | P25788 | 28 kDa |
| type-3 | | | (+1) | |
| Proteasome subunit alpha | Eutheria | PSA4_HUMAN | P25789 | 29 kDa |
| type-4 | | | | |
| Proteasome subunit alpha | Eutheria | PSA5_HUMAN | P28066 | 26 kDa |
| type-5 | | | (+1) | |
| Proteasome subunit alpha | Eutheria | PSA6_HUMAN | P60900 | 27 kDa |
| type-6 | | | | |
| Proteasome subunit alpha | Homo sapiens | PSA7_HUMAN | O14818 | 28 kDa |
| type-7 | | | | |
| Proteasome subunit beta | Homo sapiens | PSB3_HUMAN | P49720 | 23 kDa |
| type-3 | | | | |
| Proteasome subunit beta | Hominidae | PSB5_HUMAN | P28074 | 28 kDa |
| type-5 | | | | |
| Protein AHNAK2 | Homo sapiens | AHNK2_HUMAN | Q8IVF2 | 617 kDa |
| Protein AMBP | Homo sapiens | AMBP_HUMAN | P02760 | 39 kDa |
| Protein arginine N- | Catarrhini | ANM5_HUMAN | O14744 | 73 kDa |
| methyltransferase 5 | | | | |
| Protein diaphanous homolog | Homo sapiens | DIAP1_HUMAN | O60610 | 141 kDa |
| 1 | | | (+2) | |
| Protein disulfide-isomerase | Homo sapiens | PDIA1_HUMAN | P07237 | 57 kDa |
| Protein disulfide-isomerase | Hominidae | PDIA3_HUMAN | P30101 | 57 kDa |
| A3 | | | | |
| Protein disulfide-isomerase | Homo sapiens | PDIA4_HUMAN | P13667 | 73 kDa |
| A4 | | | | |
| Protein disulfide-isomerase | Homo sapiens | PDIA6_HUMAN | Q15084 | 48 kDa |
| A6 | | | | |
| Protein DJ-1 | Hominoidea | PARK7_HUMAN | Q99497 | 20 kDa |
| Protein FAM49B | Eutheria | FA49B_HUMAN | Q9NUQ9 | 37 kDa |
| Protein flightless-1 homolog | Homo sapiens | FLII_HUMAN | Q13045 | 145 kDa |
| Protein kinase C inhibitor-2 | Homo sapiens | Q8WYJ5_HUMAN | Q8WYJ5 | 14 kDa |
| | | | (+1) | |
| Protein kinase, cGMP- | Theria | Q5JP05_HUMAN | Q5JP05 | 32 kDa |
| dependent, type I | | | | |
| Protein phosphatase 1G | Homininae | PPM1G_HUMAN | O15355 | 59 kDa |
| Protein S100-A10 | Theria | S10AA_HUMAN | P60903 | 11 kDa |
| Protein SEC13 homolog | Homo sapiens | SEC13_HUMAN | P55735 | 36 kDa |
| Protein SET | Homininae | SET_HUMAN | Q01105 | 33 kDa |
| Protein transport protein | Homo sapiens | SC24C_HUMAN | P53992 | 118 kDa |
| Sec24C | | | | |
| Protein-glutamine gamma- | Homo sapiens | TGM2_HUMAN | P21980 | 77 kDa |
| glutamyltransferase 2 | | | | |
| Puromycin-sensitive | Homininae | PSA_HUMAN | P55786 | 103 kDa |
| aminopeptidase | | | | |
| Putative pre-mRNA-splicing | Homininae | DHX15_HUMAN | O43143 | 91 kDa |
| factor ATP-dependent RNA | | | | |
| helicase DHX15 | | | | |
| Putative RNA-binding | Simiiformes | LC7L2_HUMAN | Q9Y383 | 47 kDa |
| protein Luc7-like 2 | | | | |
| PWWP domain-containing | Homo sapiens | MUML1_HUMAN | Q5H9M0 | 79 kDa |
| protein MUM1L1 | | | | |
| Pyridoxal kinase | Homo sapiens | PDXK_HUMAN | O00764 | 35 kDa |
| Pyrroline-5-carboxylate | Homo sapiens | A6NFM2_HUMAN | A6NFM2 | 33 kDa |
| reductase | | | (+3) | |
| Pyruvate kinase isozymes | Homininae | KPYM_HUMAN | P14618 | 58 kDa |
| M1/M2 | | | | |
| Rab GDP dissociation | Hominoidea | GDIB_HUMAN | P50395 | 51 kDa |
| inhibitor beta | | | (+1) | |
| RAB1B protein | Homo sapiens | Q6FIG4_HUMAN | Q6FIG4 | 22 kDa |
| | | | (+1) | |
| Ran-specific GTPase- | Hominidae | RANG_HUMAN | P43487 | 23 kDa |
| activating protein | | | | |
| Ras GTPase-activating | Homo sapiens | NGAP_HUMAN | Q9UJF2 | 129 kDa |
| protein nGAP | | | | |
| Ras GTPase-activating | Homo sapiens | G3BP1_HUMAN | Q13283 | 52 kDa |
| protein-binding protein 1 | | | (+2) | |
| Ras GTPase-activating-like | Homo sapiens | IQGA1_HUMAN | P46940 | 189 kDa |
| protein IQGAP1 | | | | |
| Ras-related protein Rab-14 | Euteleostomi | RAB14_HUMAN | P61106 | 24 kDa |
| Ras-related protein Rab-21 | Catarrhini | RAB21_HUMAN | Q9UL25 | 24 kDa |
| Ras-related protein Rab-5C | Catarrhini | RAB5C_HUMAN | P51148 | 23 kDa |
| Ras-related protein Rab-7a | Theria | RAB7A_HUMAN | P51149 | 23 kDa |
| Receptor-type tyrosine- | Homo sapiens | PTPRS_HUMAN | Q13332 | 217 kDa |
| protein phosphatase S | | | | |
| Replication factor C subunit | Hominoidea | RFC3_HUMAN | P40938 | 41 kDa |
| 3 | | | | |
| Reticulon-4 | Homo sapiens | RTN4_HUMAN | Q9NQC3 | 130 kDa |
| Rho GTPase-activating | Homo sapiens | RHG32_HUMAN | A7KAX9 | 231 kDa |
| protein 32 | | | | |
| Rho guanine nucleotide | Homo sapiens | ARHG1_HUMAN | Q92888 | 102 kDa |
| exchange factor 1 | | | | |
| Ribonuclease inhibitor | Homo sapiens | RINI_HUMAN | P13489 | 50 kDa |
| Ribonucleoside-diphosphate | Homo sapiens | RIR2_HUMAN | P31350 | 45 kDa |
| reductase subunit M2 | | | | |
| Ribosome biogenesis protein | Homo sapiens | BRX1_HUMAN | Q8TDN6 | 41 kDa |
| BRX1 homolog | | | | |
| Ribosome-binding protein 1 | Homo sapiens | RRBP1_HUMAN | Q9P2E9 | 152 kDa |
| ROBO2 isoform a | Homo sapiens | Q19AB5_HUMAN | Q19AB5 | 153 kDa |
| | | | (+1) | |
| RPL14 protein | Homo sapiens | Q6IPH7_HUMAN | Q6IPH7 | 24 kDa |
| RuvB-like 1 | Eutheria | RUVB1_HUMAN | Q9Y265 | 50 kDa |
| RuvB-like 2 | Catarrhini | RUVB2_HUMAN | Q9Y230 | 51 kDa |
| Sacsin | Homo sapiens | SACS_HUMAN | Q9NZJ4 | 521 kDa |
| Sarcoplasmic/endoplasmic | Homo sapiens | AT2A2_HUMAN | P16615 | 115 kDa |
| reticulum calcium ATPase 2 | | | | |
| Sec1 family domain- | Homo sapiens | SCFD1_HUMAN | Q8WVM8 | 72 kDa |
| containing protein 1 | | | | |
| SEC23-interacting protein | Homo sapiens | S23IP_HUMAN | Q9Y6Y8 | 111 kDa |
| Secretory carrier-associated | Homininae | SCAM3_HUMAN | O14828 | 38 kDa |
| membrane protein 3 | | | | |
| Septin-2 | Catarrhini | SEPT2_HUMAN | Q15019 | 41 kDa |
| Sequestosome-1 | Homo sapiens | SQSTM_HUMAN | Q13501 | 48 kDa |
| Serine | Homo sapiens | GLYM_HUMAN | P34897 | 56 kDa |
| hydroxymethyltransferase, | | | (+1) | |
| mitochondrial | | | | |
| Serine palmitoyltransferase 2 | Homo sapiens | SPTC2_HUMAN | O15270 | 63 kDa |
| Serine/arginine-rich splicing | Amniota | SRSF3_HUMAN | P84103 | 19 kDa |
| factor 3 | | | | |
| Serine/threonine-protein | Homo sapiens | NEK9_HUMAN | Q8TD19 | 107 kDa |
| kinase Nek9 | | | | |
| Serine/threonine-protein | Homo sapiens | OXSR1_HUMAN | O95747 | 58 kDa |
| kinase OSR1 | | | | |
| Serine/threonine-protein | Homo sapiens | PAK2_HUMAN | Q13177 | 58 kDa |
| kinase PAK 2 | | | | |
| Serine/threonine-protein | Simiiformes | 2AAA_HUMAN | P30153 | 65 kDa |
| phosphatase 2A 65 kDa | | | | |
| regulatory subunit A alpha | | | | |
| isoform | | | | |
| Serine/threonine-protein | Homo sapiens | PTPA_HUMAN | Q15257 | 41 kDa |
| phosphatase 2A activator | | | | |
| Serine/threonine-protein | Theria | PP2AA_HUMAN | P67775 | 36 kDa |
| phosphatase 2A catalytic | | | (+1) | |
| subunit alpha isoform | | | | |
| Serine--tRNA ligase, | Homo sapiens | SYSC_HUMAN | P49591 | 59 kDa |
| cytoplasmic | | | (+1) | |
| Serotransferrin | Homo sapiens | TRFE_HUMAN | P02787 | 77 kDa |
| | | | (+1) | |
| Serpin B6 | Homo sapiens | SPB6_HUMAN | P35237 | 43 kDa |
| Serum albumin | Homo sapiens | ALBU_HUMAN | P02768 | 69 kDa |
| Serum albumin | Bos taurus | ALBU_BOVIN | P02769 | 69 kDa |
| Serum amyloid A-4 protein | Homo sapiens | SAA4_HUMAN | P35542 | 15 kDa |
| Serum amyloid P-component | Homo sapiens | SAMP_HUMAN | P02743 | 25 kDa |
| Serum | Homo sapiens | PON1_HUMAN | P27169 | 40 kDa |
| paraoxonase/arylesterase 1 | | | | |
| SH2 domain-containing | Homo sapiens | SHE_HUMAN | Q5VZ18 | 54 kDa |
| adapter protein E | | | | |
| SH3 domain-containing | Homo sapiens | SH3K1_HUMAN | Q96B97 | 73 kDa |
| kinase-binding protein 1 | | | (+1) | |
| Sialic acid synthase | Homininae | SIAS_HUMAN | Q9NR45 | 40 kDa |
| Signal peptide, CUB and | Homo sapiens | SCUB2_HUMAN | Q9NQ36 | 110 kDa |
| EGF-like domain-containing | | | | |
| protein 2 | | | | |
| Signal recognition particle 54 | Eutheria | SRP54_HUMAN | P61011 | 56 kDa |
| kDa protein | | | | |
| Signal recognition particle 72 | Homo sapiens | SRP72_HUMAN | O76094 | 75 kDa |
| kDa protein | | | | |
| Small nuclear | Eutheria | RSMB_HUMAN | P14678 | 25 kDa |
| ribonucleoprotein-associated | | | (+4) | |
| proteins B and B′ | | | | |
| Sodium/potassium- | Homo sapiens | AT1A1_HUMAN | P05023 | 113 kDa |
| transporting ATPase subunit | | | | |
| alpha-1 | | | | |
| Solute carrier family 2, | Homininae | GTR1_HUMAN | P11166 | 54 kDa |
| facilitated glucose transporter | | | (+1) | |
| member 1 | | | | |
| Sorbitol dehydrogenase | Homo sapiens | DHSO_HUMAN | Q00796 | 38 kDa |
| Sorting nexin-6 | Homo sapiens | SNX6_HUMAN | Q9UNH7 | 47 kDa |
| SP-A receptor subunit SP- | Homo sapiens | Q5QD01_HUMAN | Q5QD01 | 181 kDa |
| R210 alphaS | | | (+1) | |
| Spectrin alpha chain, brain | Homo sapiens | SPTA2_HUMAN | Q13813 | 285 kDa |
| Spectrin beta chain, brain 1 | Homininae | SPTB2_HUMAN | Q01082 | 275 kDa |
| Spectrin repeat containing, | Homo sapiens | Q5JV23_HUMAN | Q5JV23 | 1005 kDa |
| nuclear envelope 1 | | | (+1) | |
| Sphingosine-1-phosphate | Homo sapiens | SGPL1_HUMAN | O95470 | 64 kDa |
| lyase 1 | | | | |
| Spliceosome RNA helicase | Theria | DX39B_HUMAN | Q13838 | 49 kDa |
| DDX39B | | | | |
| Splicing factor 3B subunit 3 | Eutheria | SF3B3_HUMAN | Q15393 | 136 kDa |
| Splicing factor, proline- and | Eutheria | SFPQ_HUMAN | P23246 | 76 kDa |
| glutamine-rich | | | (+1) | |
| Staphylococcal nuclease | Homo sapiens | SND1_HUMAN | Q7KZF4 | 102 kDa |
| domain-containing protein 1 | | | | |
| STIP1 protein | Homo sapiens | Q3ZCU9_HUMAN | Q3ZCU9 | 68 kDa |
| Stomatin-like protein 2 | Hominidae | STML2_HUMAN | Q9UJZ1 | 39 kDa |
| Stress-70 protein, | Homo sapiens | GRP75_HUMAN | P38646 | 74 kDa |
| mitochondrial | | | | |
| Structural maintenance of | Homo sapiens | SMHD1_HUMAN | A6NHR9 | 226 kDa |
| chromosomes flexible hinge | | | | |
| domain-containing protein 1 | | | | |
| Superoxide dismutase [Cu—Zn] | Homininae | SODC_HUMAN | P00441 | 16 kDa |
| Talin-1 | Homo sapiens | TLN1_HUMAN | Q9Y490 | 270 kDa |
| T-complex protein 1 subunit | Catarrhini | TCPA_HUMAN | P17987 | 60 kDa |
| alpha | | | | |
| T-complex protein 1 subunit | Catarrhini | TCPB_HUMAN | P78371 | 57 kDa |
| beta | | | | |
| T-complex protein 1 subunit | Hominoidea | TCPD_HUMAN | P50991 | 58 kDa |
| delta | | | | |
| T-complex protein 1 subunit | Catarrhini | TCPE_HUMAN | P48643 | 60 kDa |
| epsilon | | | | |
| T-complex protein 1 subunit | Hominoidea | TCPZ_HUMAN | P40227 | 58 kDa |
| zeta | | | | |
| Testin | Hominidae | TES_HUMAN | Q9UGI8 | 48 kDa |
| Thioredoxin reductase 1, | Homo sapiens | TRXR1_HUMAN | Q16881 | 71 kDa |
| cytoplasmic | | | | |
| Thioredoxin-dependent | Homininae | PRDX3_HUMAN | P30048 | 28 kDa |
| peroxide reductase, | | | | |
| mitochondrial | | | | |
| THO complex subunit 4 | Homo sapiens | THOC4_HUMAN | Q86V81 | 27 kDa |
| Threonine--tRNA ligase, | Homo sapiens | SYTC_HUMAN | P26639 | 83 kDa |
| cytoplasmic | | | | |
| Tight junction protein ZO-2 | Homo sapiens | ZO2_HUMAN | Q9UDY2 | 134 kDa |
| Titin | Homo sapiens | TITIN_HUMAN | Q8WZ42 | 3816 kDa |
| Transcription elongation | Homo sapiens | ELOA1_HUMAN | Q14241 | 90 kDa |
| factor B polypeptide 3 | | | | |
| Transcription factor p65 | Homininae | TF65_HUMAN | Q04206 | 60 kDa |
| Transcription intermediary | Homininae | TIF1B_HUMAN | Q13263 | 89 kDa |
| factor 1-beta | | | | |
| Transferrin receptor protein 1 | Homo sapiens | TFR1_HUMAN | P02786 | 85 kDa |
| Transgelin-2 | Catarrhini | TAGL2_HUMAN | P37802 | 22 kDa |
| Transitional endoplasmic | Euarchontoglires | TERA_HUMAN | P55072 | 89 kDa |
| reticulum ATPase | | | | |
| Transketolase | Homo sapiens | TKT_HUMAN | P29401 | 68 kDa |
| Translational activator GCN1 | Homo sapiens | GCN1L_HUMAN | Q92616 | 293 kDa |
| Translin | Euarchontoglires | TSN_HUMAN | Q15631 | 26 kDa |
| Translocon-associated | Homo sapiens | SSRA_HUMAN | P43307 | 32 kDa |
| protein subunit alpha | | | | |
| Transmembrane emp24 | Homo sapiens | TMEDA_HUMAN | P49755 | 25 kDa |
| domain-containing protein 10 | | | | |
| Transportin-1 | Homininae | TNPO1_HUMAN | Q92973 | 102 kDa |
| Trifunctional enzyme subunit | Homo sapiens | ECHA_HUMAN | P40939 | 83 kDa |
| alpha, mitochondrial | | | | |
| Triosephosphate isomerase | Homininae | TPIS_HUMAN | P60174 | 31 kDa |
| tRNA (cytosine(34)-C(5))- | Homo sapiens | NSUN2_HUMAN | Q08J23 | 86 kDa |
| methyltransferase | | | | |
| tRNA-splicing ligase RtcB | Eutheria | RTCB_HUMAN | Q9Y3I0 | 55 kDa |
| homolog | | | | |
| Tropomodulin-3 | Homo sapiens | TMOD3_HUMAN | Q9NYL9 | 40 kDa |
| Tropomyosin 3 | Eutheria | Q5VU58_HUMAN | Q5VU58 | 29 kDa |
| Tryptophan--tRNA ligase, | Hominidae | SYWC_HUMAN | P23381 | 53 kDa |
| cytoplasmic | | | | |
| Tubulin alpha-1C chain | Hominidae | TBA1C_HUMAN | Q9BQE3 | 50 kDa |
| Tubulin alpha-4A chain | Eutheria | TBA4A_HUMAN | P68366 | 50 kDa |
| Tubulin beta chain | Amniota | TBB5_HUMAN | P07437 | 50 kDa |
| Tubulin beta-4B chain | Theria | TBB4B_HUMAN | P68371 | 50 kDa |
| | | | (+1) | |
| Tubulin beta-6 chain | Hominoidea | TBB6_HUMAN | Q9BUF5 | 50 kDa |
| Tyrosine-protein phosphatase | Homo sapiens | PTN13_HUMAN | Q12923 | 277 kDa |
| non-receptor type 13 | | | | |
| U5 small nuclear | Hominidae | U520_HUMAN | O75643 | 245 kDa |
| ribonucleoprotein 200 kDa | | | | |
| helicase | | | | |
| Ubiquitin carboxyl-terminal | Homininae | UBP5_HUMAN | P45974 | 96 kDa |
| hydrolase 5 | | | | |
| Ubiquitin carboxyl-terminal | Homo sapiens | UBP8_HUMAN | P40818 | 128 kDa |
| hydrolase 8 | | | | |
| Ubiquitin carboxyl-terminal | Hominidae | UCHL1_HUMAN | P09936 | 25 kDa |
| hydrolase isozyme L1 | | | | |
| Ubiquitin thioesterase | Eutheria | OTUB1_HUMAN | Q96FW1 | 31 kDa |
| OTUB1 | | | | |
| Ubiquitin-60S ribosomal | Tetrapoda | RL40_HUMAN | P62987 | 15 kDa |
| protein L40 | | | (+3) | |
| Ubiquitin-associated protein | Homo sapiens | UBP2L_HUMAN | Q14157 | 115 kDa |
| 2-like | | | | |
| Ubiquitin-like modifier- | Homo sapiens | UBA1_HUMAN | P22314 | 118 kDa |
| activating enzyme 1 | | | | |
| UDP-glucose 6- | Hominidae | UGDH_HUMAN | O60701 | 55 kDa |
| dehydrogenase | | | | |
| UDP-glucose: glycoprotein | Homo sapiens | UGGG1_HUMAN | Q9NYU2 | 177 kDa |
| glucosyltransferase 1 | | | | |
| UDP-N-acetylhexosamine | Homo sapiens | UAP1_HUMAN | Q16222 | 59 kDa |
| pyrophosphorylase | | | | |
| UMP-CMP kinase | Homo sapiens | KCY_HUMAN | P30085 | 22 kDa |
| | | | (+2) | |
| Unc-45 homolog A | Homo sapiens | A8K6F7_HUMAN | A8K6F7 | 102 kDa |
| (C. elegans), isoform CRA_a | | | (+1) | |
| Unconventional myosin-Ic | Homo sapiens | MYO1C_HUMAN | O00159 | 122 kDa |
| Unconventional myosin-Ie | Homo sapiens | MYO1E_HUMAN | Q12965 | 127 kDa |
| Unconventional myosin-VI | Homo sapiens | MYO6_HUMAN | Q9UM54 | 150 kDa |
| Uridine 5′-monophosphate | Homo sapiens | UMPS_HUMAN | P11172 | 52 kDa |
| synthase | | | | |
| Uridine phosphorylase 1 | Homininae | UPP1_HUMAN | Q16831 | 34 kDa |
| UTP--glucose-1-phosphate | Hominidae | UGPA_HUMAN | Q16851 | 57 kDa |
| uridylyltransferase | | | | |
| UV excision repair protein | Homininae | RD23B_HUMAN | P54727 | 43 kDa |
| RAD23 homolog B | | | | |

### Example 11—Summary of the Methods of the Present Disclosure Versus Commercially Available Kits

As can be seen from the results above, the methods of the present disclosure provide a mechanism to isolate biologically relevant circulating cell free nucleic acids along with associated cellular components, such as lipids and polypeptides. Both the nucleic acid and cellular components can be used to determine the condition of a subject and to analyze a variety of biomarkers. Furthermore the methods of the present disclosure selectively isolate active genomic regions, selecting for the most relevant nucleic acid targets for analysis. In addition to increased purity and yield, the methods of the present disclosure also use significantly smaller volumes of sample starting material for the analysis. In summary, the methods of the present disclosure provide significant advantage over the methods known in the prior art. A comparison of the characteristic of the methods of the present disclosure versus the prior art is provided in Table 11.

| | Methods of the Prior | |
|---|---|---|
| | Art | Present Disclosure |
| Main Principle | Random, blinded | Selective enrichment of |
| | isolation of nucleic acid | lipid/polypeptide associated |
| | | nucleic acid complexes |
| | | (active genomic regions) |
| Genomic | Poor-Fair | Excellent |
| Representation | | |
| of Active Gene | | |
| (eg, oncogenes) | | |
| Starting | Large Volume | Very Small Volume |
| Material | | |
| (eg, plasma) | | |
| Yield | Low | High |
| Functionality | No | Yes |
| Studies | | |
| ID Associated | No | Yes |
| Biomarkers | | |
| Sensitivity/ | Low (solely depends on | High |
| Specificity | extraction efficiency) | |

What is claimed:

1. (canceled)

2. (canceled)

3. (canceled)

4. (canceled)

5. (canceled)

6. (canceled)

7. (canceled)

8. (canceled)

9. A method for analysis of a cell free complex comprising circulating, cell-free nucleic acid and associated cellular components, the method comprising isolating the cell free complex by subjecting a sample containing the cell free complex to centrifugation, optionally further processing the cell free complex and analyzing the nucleic acid, associated cellular components or a combination of the foregoing to determine a characteristic of the nucleic acid, associated cellular components or a combination of the foregoing.

10. The method of claim 9, wherein the method further comprises isolating at least one cell-free nucleic acid from the complex.

11. The method of claim 10, wherein the cell-free nucleic acid is associated with a cellular component.

12. The method of claim 12, wherein the cellular component is such as a polypeptide or a lipid.

13. The method of claim 9, wherein the cell-free nucleic acid is DNA or RNA.

14. The method of claim 9, wherein the centrifugation is density gradient ultracentrifugation.

15. (canceled)

16. The method of claim 15, wherein the density gradient has a buoyant density from 1.3 to 1.45 g/cm³.

17. The method of claim 9, wherein the characteristic is a nucleic acid characteristic, a polypeptide characteristic or a lipid characteristic.

18. The method of claim 9, wherein the nucleic acid characteristic is presence of a mutation, presence of a polymorphism, methylation state, concentration of a nucleic acid, level of expression of a nucleic acid, a nucleic acid profile or a combination of the foregoing.

19. The method of claim 9, wherein the polypeptide characteristic is presence of a mutation, the presence of post-translational modification, presence of insertions or deletions, concentration of a polypeptide, the expression level of a polypeptide, a polypeptide profile or a combination of the foregoing.

20. The method of claim 9, wherein the lipid characteristic is presence of altered forms of a lipid, the presence of a lipid modification, the concentration, the expression level of a lipid, the lipid profile or a combination of the foregoing.

21. A method for determining a biologically relevant profile of a subject, the method comprising isolating a cell free complex comprising circulating, cell-free nucleic acid and associated cellular components by subjecting a sample containing the cell free complex to centrifugation, optionally further processing the cell free complex and identifying a plurality of nucleic acids or associated cellular components to produce the profile.

22. The method of claim 22 further comprising comparing the profile of the subject to a corresponding profile indicative of a disease state and determining the subject is suffering from or at risk for a particular disease or condition if the subject profile contains one or more characteristics of the corresponding profile indicative of a disease state

23. The method of claim 22, wherein a plurality of nucleic acids are identified to create a genetic profile.

24. The method of claim 22, wherein the associated cellular components are a polypeptide or a lipid.

25. The method of claim 24, wherein a plurality of polypeptides are identified to create a polypeptide profile.

26. The method of claim 24, wherein a plurality of lipids are identified to create a lipid profile.

27. The method of claim 21, wherein the method further comprises isolating at least one cell-free nucleic acid from the complex.

28. The method of claim 27, wherein the cell-free nucleic acid is associated with a cellular component.

29. The method of claim 28, wherein the cellular component is a polypeptide or a lipid.

30. The method of claim 22, wherein the cell-free nucleic acid is DNA or RNA.

31. The method of claim 22, wherein the centrifugation is density gradient ultracentrifugation.

32. (canceled)

33. The method of claim 32, wherein the density gradient has a buoyant density from 1.3 to 1.45 g/cm³.

34. The method of claim 22, wherein the corresponding profile is a disease fingerprint.
